(19) United States
(12) Patent Application Publication
(10) Pub. No.: US 2026/0078211 A1
(43) Pub. Date: Mar. 19, 2026

(54) A SOLID PARTICLE OF ORGANOSILICON-FUNCTIONAL CO-POLYMER, MANUFACTURING PROCESS THEREOF, AND COSMETICS COMPRISING SAID SOLID PARTICLE

(71) Applicants: DOW TORAY CO., LTD., Shinagawa-Ku, Tokyo (JP); DOW SILICONES CORPORATION, Midland, MI (US)

(72) Inventors: Tatsuo SOUDA, Ichihara-shi, Chiba (JP); Momoko OFUCHI, Ichihara-shi, Chiba (JP); Tsunehito SUGIURA, Ichihara-shi, Chiba (JP); Seiji HORI, Ichihara-shi, Chiba (JP); Hidenori MATSUSHIMA, Ichihara-shi, Chiba (JP); Kouichi OZAKI, Ichihara-shi, Chiba (JP); Takeshi YOSHIZAWA, Ichihara-shi, Chiba (JP); Isabelle VAN REETH, Seneffe (BE); Xinyan BAO, Shanghai (CN)

(21) Appl. No.: 19/134,155

(22) PCT Filed: Nov. 30, 2022
(86) PCT No.: PCT/CN2022/135206

§ 371 Date: May 29, 2025

(51) Int. Cl. C08F 283/12; A61K 8/91; C08F 290/06

(52) U.S. Cl. CPC C08F 283/124; A61K 8/91; C08F 290/068

(57) ABSTRACT

Provided is a solid particle consisting essentially of organosilicon-functional co-polymer polymerized from a monomer composition. The monomer composition consists essentially of (A) one or more unsaturated polymerizable monomer(s) having at least one organosilicon functionality and one polymerizable group in the molecule and (B) one or more unsaturated polymerizable monomer(s) containing no or one silicon atom and having one polymerizable group in the molecule. The mass ratio of monomer (A) to monomer (B) ranges from 35:65 to 70:30 in the monomer composition. The long size in three directions for the solid primary particle ranges from 0.1 to 5,000 μm. A manufacturing process of the solid particle, use of the solid particle as a cosmetic ingredient, as well as a cosmetic composition comprising the solid particle and the production method thereof are also provided.

### TECHNOLOGICAL FIELD OF THE PRESENT INVENTION

The present invention relates to a solid particle of organosilicon-functional co-polymer having superior solubility in cosmetic liquid medium and useful as cosmetic ingredient. This invention also relates to the manufacturing process thereof, and cosmetic composition comprising the solid particle.

### BACKGROUND ART

Attempts have been made to improve water resistance and sebum resistance of cosmetic materials in order to improve (long-lasting) the cosmetic retainability of cosmetic materials, and particularly of makeup cosmetic materials. Use of an organopolysiloxane-containing polymer in a cosmetic material for the purpose of improving water resistance, etc. of the cosmetic material is conventionally known (for example, see Patent Document 1). However, commercial silicone acrylates are available as either solution form or dispersion form. Therefore, this product form with additives and/or solvents, when utilizing to each downstream formulation with other various additives, restricts carriers or reduces freedom of choice and the variety of formulation design. For example, when a silicone acrylate has isododecane as a solvent, the resulting cosmetic formulations and/or commercial products, such as lipsticks, have isododecane in it too. Lipsticks formulated with isododecane give unpleasant feeling to users because isododecane acts as a plasticizer.

In addition, such a carrier fluid has low flash point, and careful transport and handling are thus required.

There are known approaches found in prior documents to overcome these disadvantages.

As an exemplary approach, commercial silicone acrylate isododecane solution was evaporated and pulverized to crash by hand to prepare silicone acrylate granule. Such solid could be dispersed or resolved in a natural cosmetic oil (see Patent Document 2). However, it required hard work to realize hand crush, and the process realized such hand crush of solid silicone acrylate could not be applied to mass production. In addition, the pulverized silicone acrylate solids required high shear mixing, long time and heating to be completely solved. Therefore, in the market, only solution type products are available. Attempt with ball mill was known (see Patent Document 1). In the trial, employed silicone acrylate had low glass temperature so as to be soft. Therefore, aggregation and/or accumulation was caused by share exotherm during milling (Comparative Example 3). As a result, obtained solid was not easily solved in cosmetic oils. Also, the low silicone moiety in the polymer resulted in low oil repellency not to be allowed for cosmetic applications. For those prior arts, the appropriate features for use of solid silicone acrylates were not described. Users using solid silicone acrylates desire an easy soluble solid silicone acrylate for mass production. Then, inventors investigated the proper physical properties that provides solid silicone acrylate meeting both mass production and easy solubility.

On the other hand, powdering process was also practiced. Silicone acrylate emulsion was prepared first. And next, powder was obtained by a spray drier (see Patent Document 3). However, employed acryl silicone as a starting material has 2 or more (meth)acrylic groups in one monomer which enables crosslinking. Crosslinked silicone acrylates have less aggregation because they are no longer thermoplastic polymers. Thus, such polymer was easily processed even at high temperature during drying to make fine powder. However, such powder polymer could not be solved in any oils due to their thermosetting property.

### Prior Art Documents

Patent Document1: Japanese Unexamined Patent Application Publication No. 2000-63225
Patent Document 2: US Unexamined Patent US20160374930
Patent Document 4: Germany Unexamined Patent DE102007058713

### SUMMARY OF THE INVENTION

### Problem to be Solved by the Invention

In the silicone acrylate technology field, easy soluble solid particle is not well known. There are only a few prior arts comprising elastic silicone acrylates including crosslinker, but such elastic particle can't be solved in cosmetic oils. Although there is an example found in prior patent (see Patent Document 3), where silicone acrylate powder consisting of 20% of silicone monomer was prepared via ball mill, the inventors realized that particle was hardly prepared from such material due to its tackiness and the particle required heating at higher temperature and high shear mixing to completely solve in isododecane. Therefore, manufacturable molecular design and much easier soluble solid silicone acrylate were desired.

Moreover, current offering silicone acrylate are mainly offered in dispersion form with active level from 30˜50%. Cosmetic manufactures don't have freedom to select their preferred carrier/solvent or carrier solvent blends as the possibility to use the neat silicone acrylate in formulations. In most case, these silicone acrylates copolymers are available under dispersion form delivered from volatile silicone fluids or hydrocarbons. In the case of volatile hydrocarbons such as isododecane, the final dispersion is classified as dangerous good (flammable) resulting in significantly higher transportation and storage cost. According to prior art, the only way to get a neat solventless silicone acrylate is to evaporate the carrier/solvent present in the dispersion resulting into a bulk solid which is very difficult to re-disperse in the solvent of choice requiring high heat and high shear.

For the reasons above, an easy to disperse silicone acrylate powder is highly desired to increase the flexibility in the carrier choice as well as the possibility to make new formulation format; loose powder, pressed powders, which are difficult to make using silicone acrylate dispersion.

Accordingly, it is an object of the present invention to provide a solid particle of organosilicon-functional co-polymer that is soluble in cosmetic solvents.

Further, it is other objects of the present invention to provide the manufacturing process and use of said solid particle, as well as a cosmetic composition comprising said solid particle and the production method of said cosmetic composition.

### Means for Solving the Problem

As a result of extensive research, the present inventors discovered that a solvent-soluble solid particle of an organosilicon-functional co-polymer could be formed by polymerization from a specific monomer composition and followed specific solid process. The present invention is a product of this discovery.

One aspect of the present invention is a solid particle consisting essentially of organosilicon-functional co-polymer polymerized from a monomer composition consisting essentially of (A) one or more unsaturated polymerizable monomer(s) having at least one organosilicon functionality and one polymerizable group in the molecule and (B) one or more unsaturated polymerizable monomer(s) containing no or one silicon atom and having one polymerizable group in the molecule, wherein the mass ratio for said monomers (A) and (B) is in a range from 35:65 to 70:30 in said monomer composition, and the long size in three directions for the solid primary particle ranges from 0.1 to 5,000 μm.

In some embodiments, the glass transition point (Tg) which is calculated from a FOX equation of said solid particle preferably ranges from 35 to 120° C.

In some embodiments, said unsaturated polymerizable monomer (A) is preferably at least one selected from one represented by any of following formula (A-1) to (A-7):

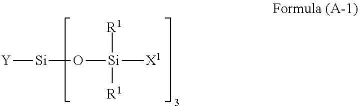

{In this formula, Y is a radically polymerizable organic group, R¹ is an alkyl or aryl group having from 1 to 10 carbon atoms, and X¹ is a silylalkyl group represented by the following formula where i=1.

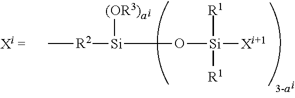

(In this formula, R¹ is the same as above, R² is an alkylene group having from 2 to 10 carbon atoms, R 3 is an alkyl group having from 1 to 10 carbon atoms, Xⁱ⁺¹ is a hydrogen atom or a group selected from the group consisting of an alkyl group having from 1 to 10 carbon atoms, an aryl group, and a silylalkyl group mentioned above, i is an integer from 1 to 10 representing the number of levels of silylalkyl groups mentioned above, and aⁱ is an integer from 0 to 3.)}

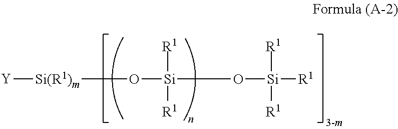

(In this formula, Y and R¹ are the same as above, m is 0, 1 or 2, and n is a number from 0 to 200 representing the average degree of polymerization.)

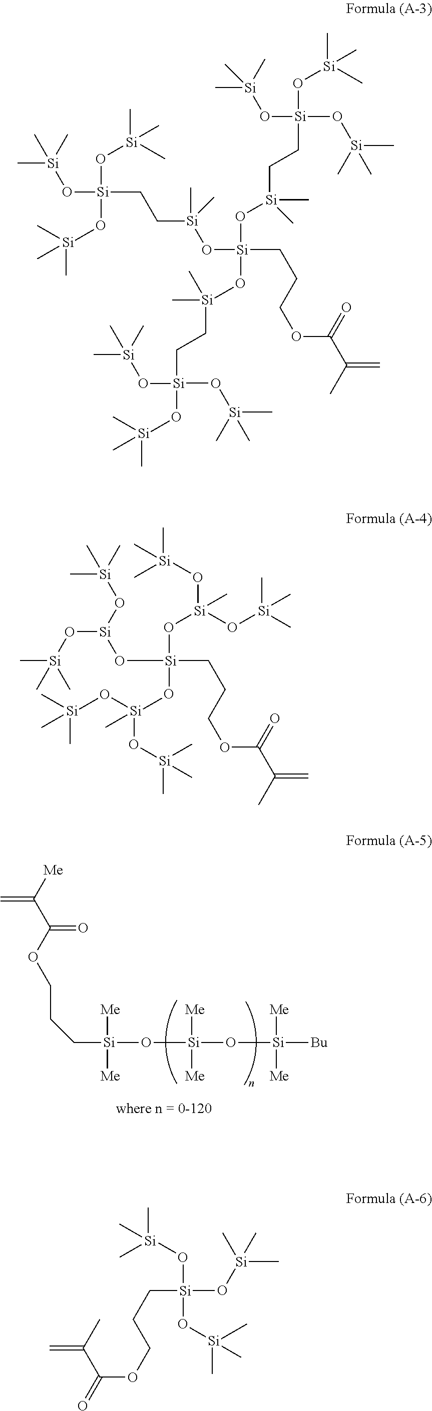

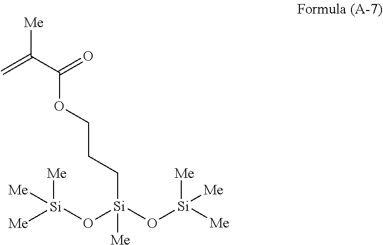 [SMILES: C=C(C)C(=O)OCCC[Si](C)(O[Si](C)(C)C)O[Si](C)(C)C]

In some embodiments, the shape of said solid particle is preferably selected from spherical particle, non-spherical particle, powder, pellet, bead, short fiber, short tube and crushed powder.

In some embodiments, the shape of said solid primary particle is preferably spherical particle having a diameter ranging from 0.1 to 5,000 μm. If the particles are aggregated, agglomerated, or flocculated, the diameter ranges from 1 to 5,000 μm.

A second aspect of the present invention is a manufacturing process of the solid particle described in the first aspect, comprising following steps:
Step (I): a step of preparing a solution or dispersion of organosilicon-functional co-polymer through polymerization reaction from a monomer composition consisting essentially of (A) an unsaturated polymerizable monomer having at least one organosilicon functionality and one polymerizable group in the molecule and (B) an unsaturated polymerizable monomer containing no or one silicon atom and having one polymerizable group in the molecule, wherein the mass ratio for said monomers (A) and (B) is in a range from 35:65 to 70:30 in said monomer composition; and
Step (II): a step of removing carrier fluid of water or solvent from the solution or dispersion of organosilicon-functional co-polymer prepared in above Step (I).

In some embodiments, the manufacturing process of the solid particle described above further comprise below step:
Step (III): a step of forming the solid particle consisting essentially of organosilicon-functional co-polymer using at least one device selected from extruder, pelletizer, mill, crusher, pulverizer, grinder, pastillator and drum flaker, following to said Step (II).

In some embodiments, the manufacturing process of the solid particle described above further comprise below step:
Step (IV): a step of classifying the coarse solid particle consisting essentially of organosilicon-functional co-polymer using at least one device selected from with screen filter, mesh, punch plate, Cyclone and Dynamic air classifier.

In some embodiments, said Step (II) is a step of spray-drying process to obtain spherical particle of said organosilicon-functional co-polymer by removing carrier fluid of water or solvent from the solution or dispersion of organosilicon-functional co-polymer through spraying said solution or dispersion.

In some embodiments, said Step (I) is a step of preparing a solution or dispersion of organosilicon-functional co-polymer through at least one liquid phase polymerization reaction selected from solution polymerization, mini-emulsion polymerization and emulsion polymerization.

A Third aspect of the present invention is use of the solid particle described in the first aspect as cosmetic ingredient, particularly as cosmetic ingredient having film-forming function in human skin and/or hair.

A fourth aspect of the present invention is a cosmetic composition comprising the solid particle described in the first aspect.

A fifth aspect of the present invention is a production method of cosmetic composition of the fourth aspect, comprising a step of preparation of solution or dispersion of the solid particle described in the first aspect into at least one cosmetic liquid medium.

In some embodiments, the cosmetic liquid medium is at least one selected from alcohols, esters, silicone fluid, hydrocarbon oils, fatty acid ester oils, liquid UV protection agent, biobased liquid, biodegradable liquid, cosmetically-acceptable solvent, and mixture thereof.

### Effect of the Invention

The present invention is able to provide a solid particle of organosilicon-functional co-polymer that is soluble in cosmetic solvents, and also to provide the manufacturing process and use of said solid particle, as well as a cosmetic composition comprising said solid particle and the production method of said cosmetic composition.

Particularly, solid particle of the present invention is a solid of silicone graft polyacrylate, where 1) wt % of silicone monomer ranges 35-70 mass %, 2) long size in three directions of the primary particle is smaller than 5,000 μm, and 3) calculated glass transition temperature ranges 35-120° C., and it enables easy soluble silicone acrylate in various oils and really manufactured. Therefore, said solid particle has superior solubility and easy handling property as cosmetic ingredient at formulating into cosmetic compositions.

### DESCRIPTION OF THE PREFERRED EMBODIMENTS

### Solvent-Soluble Solid Particle

In the present specification, “(meth)acrylic acid” refers to both acrylic acid and methacrylic acid. Similarly, “(meth)acrylate”, “(meth)acryloxy”, and “(meth)acrylamide” refer, respectively, to acrylate and methacrylate, acryloxy and methacryloxy, and acrylamide and methacrylamide. In the present invention, “cosmetic” and “cosmetic product” are used interchangeably. In the present invention, the singular form of the articles “a,” “an,” and “the” includes plural references unless indicated otherwise. In the present invention, the terms “comprise”, “comprising”, “contain”, “containing”, “include”, “including” and their variants are open claim language, i.e., are permissive of additional elements.

In the present invention, a solvent-soluble solid particle consisting essentially of organosilicon-functional co-polymer is provided. Here, the term “organosilicon-functional” means silane, silyl, organosiloxane or organocarbosiloxane functional groups bound to polymerizable group (-Y or acrylic terminal group) in monomer or obtained co-polymer. The term “consisting essentially of” and its variants are closed claim language, i.e., exclusive additional elements. For example, “a solvent-soluble solid particle consisting essentially of organosilicon-functional co-polymer” means that the solvent-soluble solid particle of the present invention does not contain, in addition to said organosilicon-functional co-polymer, materials that materially affect the basic and novel properties of the invention.

Specifically, solid particle of the present invention is a solid of silicone graft polyacrylate, where the long size in three directions for the solid particle (primary particle) is smaller than 5,000 μm. If the long size for the solid particle is larger than 5,000 μm, its solubility becomes poor and it becomes unable to resolve in various cosmetic solvents. Preferably, solid particle of the present invention has small size and large surface area, and the average of long size in three directions thereof ranges from 1 to 4,000 μm, preferably 10 to 4,000 μm, more preferably 100 to 3,000 μm, and most preferably 200 to 2,000 μm.

In some preferable embodiments, calculated glass transition temperature (Tg, calculated from a FOX equation, described below in detail) of said solid particle ranges 35-120° C. If Tg of the solid particle goes beyond the range of 35-120° C., its solubility may become poor and it may become unable to resolve in various cosmetic solvents. Preferably, Tg of said solid particle ranges 40-100° C., more preferably, 40-80° C.

For forming said solid particle of the present invention, an organosilicon-functional co-polymer is utilized. The organosilicon-functional co-polymer is polymerized from a monomer composition consisting essentially of (A) an unsaturated polymerizable monomer having at least one organosilicon functionality and one polymerizable group in the molecule and (B) an unsaturated polymerizable monomer containing no or one silicon atom and having one polymerizable group in the molecule. Here, the term “consisting essentially of” means that monomer composition for forming the organosilicon-functional co-polymer of the present invention does not contain, in addition to said monomers (A) and (B), materials that materially affect the basic and novel properties of the invention.

### [Unsaturated Polymerizable Monomer (A)]

The unsaturated polymerizable monomer (A) is an unsaturated polymerizable monomer having at least one organosilicon functionality and one polymerizable group in the molecule, and is used mainly for the purpose of introducing a polysiloxane structure to the co-polymer. In this invention, considering water/oil-repellent property and film-forming property of the organosilicon-functional co-polymer, unsaturated polymerizable monomer (A) is preferably selected from the group consisting of: carbosiloxane-dendrimer functionality derived from monomer (A-1), macromonomer-functionality derived from monomer (A-2), other linear/branched/dendrimeric siloxane/carbosiloxane functionality derived from monomers (A-3) to (A-7).

One preferred embodiment of monomer (A) used in the present invention is monomer (A1), which is represented by the following general formula (A-1).

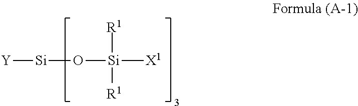

In general formula (A-1), Y is an unsaturated organic group that is radically polymerizable. Specific examples include (meth)acryloxy group-containing organic groups represented by the general formulas below, (meth) acrylamide group-containing organic groups, styryl group-containing organic groups, or alkenyl groups having from 2 to 10 carbon atoms.

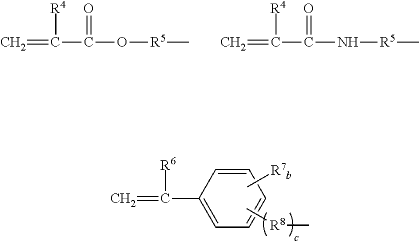

(In these formulas, R⁴ and Re are a hydrogen atom or methyl group, R⁵ and R⁸ are an alkylene group having from 1 to 10 carbon atoms, R⁷ is an alkyl group having from 1 to 10 carbon atoms, b is an integer from 0 to 4, and c is 0 or 1.) Examples of these radically polymerizable organic groups include an acryloxymethyl group, 3-acryloxypropyl group, methacryloxymethyl group, 3-methacryloxypropyl group, 4-vinylphenyl group, 3-vinylphenyl group, 4-(2-propenyl) phenyl group, 3-(2-propenyl) phenyl group, 2-(4-vinylphenyl) ethyl group, 2-(3-vinylphenyl) ethyl group, vinyl group, allyl group, methallyl group, and 5-hexenyl group. R¹ is an alkyl group or aryl group having from 1 to 10 carbon atoms. The alkyl group can be a methyl group, ethyl group, propyl group, butyl group, pentyl group, isopropyl group, isobutyl group, cyclopentyl group, or cyclohexyl group. The aryl group can be a phenyl group or a naphthyl group. Among these, a methyl group or phenyl group is preferred and a methyl group is especially preferred. X¹ is a silylalkyl group represented by the following formula where i=1.

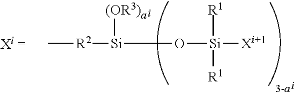

In this formula, R² is an alkylene group having from 2 to 10 carbon atoms. Examples include linear alkylene groups such as an ethylene group, propylene group, butylene group and hexylene group; and branched alkylene groups such as a methylmethylene group, methylethylene group, 1-methylpentylene group and 1,4-dimethylbutylene group. Among these, an ethylene group, methylethylene group, hexylene group, 1-methylpentylene group or 1,4-dimethylbutylene group is preferred. R³ is an alkyl group having from 1 to 10 carbon atoms. Examples include a methyl group, ethyl group, propyl group, butyl group and isopropyl group. R¹ is the same as above. Xi+1 is a hydrogen atom or a group selected from the group consisting of an alkyl group having from 1 to 10 carbon atoms, an aryl group, and a silylalkyl group mentioned above. aⁱ is an integer from 0 to 3, preferably from 0 to 2, more preferably 0 to 1, and even more preferably 0. i is an integer from 1 to 10 and represents the number of levels of silylalkyl groups mentioned above, that is, the number of repeating silylalkyl groups. Therefore, when the number of levels is 1, the carbosiloxane dendrimer in this component is represented by the following general formula.

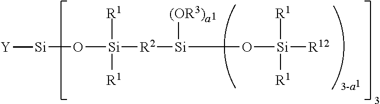

(In this formula, Y, R¹, R² and R³ are the same as above, R¹² is a hydrogen atom or the same as R¹ above, and a¹ is the same as aⁱ above except that the average total number of a¹ per molecule is from 0 to 7.) When the number of levels is 2, the carbosiloxane dendrimer in this component is represented by the following general formula.

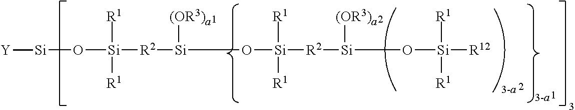

(In this formula, Y, R¹, R², R³ and R¹² are the same as above, and a¹ and a² are the same as a¹ above except that the average total number of a¹ and a² per molecule is from 0 to 25.) When the number of levels is 3, the carbosiloxane dendrimer in this component is represented by the following general formula.

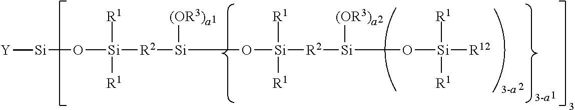

(In this formula, Y, R¹, R², R³ and R¹² are the same as above, and a¹, a² and a³ are the same as aⁱ above except that the average total number of a¹, a² and a³ per molecule is from 0 to 79.)

Examples of carbosiloxane dendrimers containing radically polymerizable organic groups in this component include the carbosiloxane dendrimers represented by the following average composition formulas.

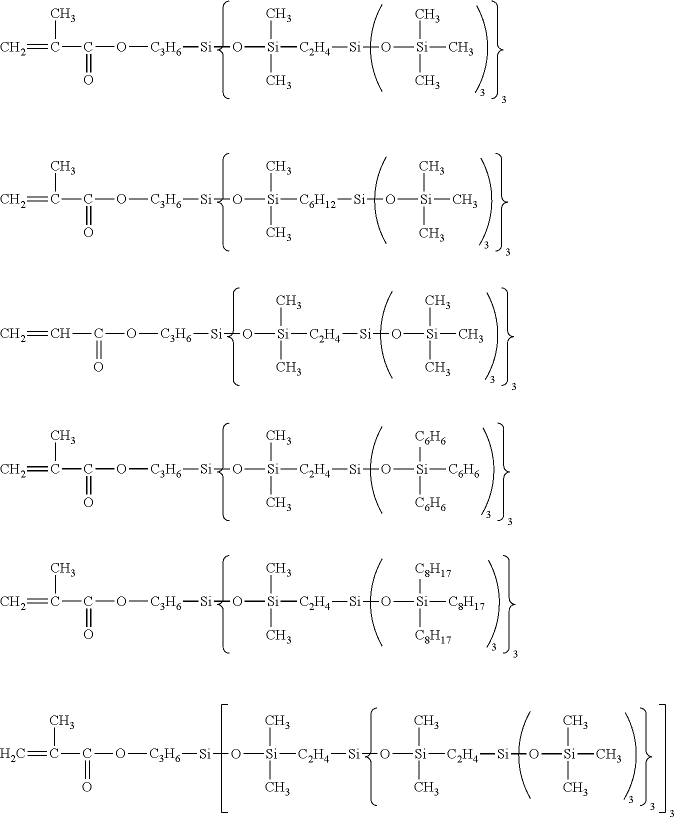

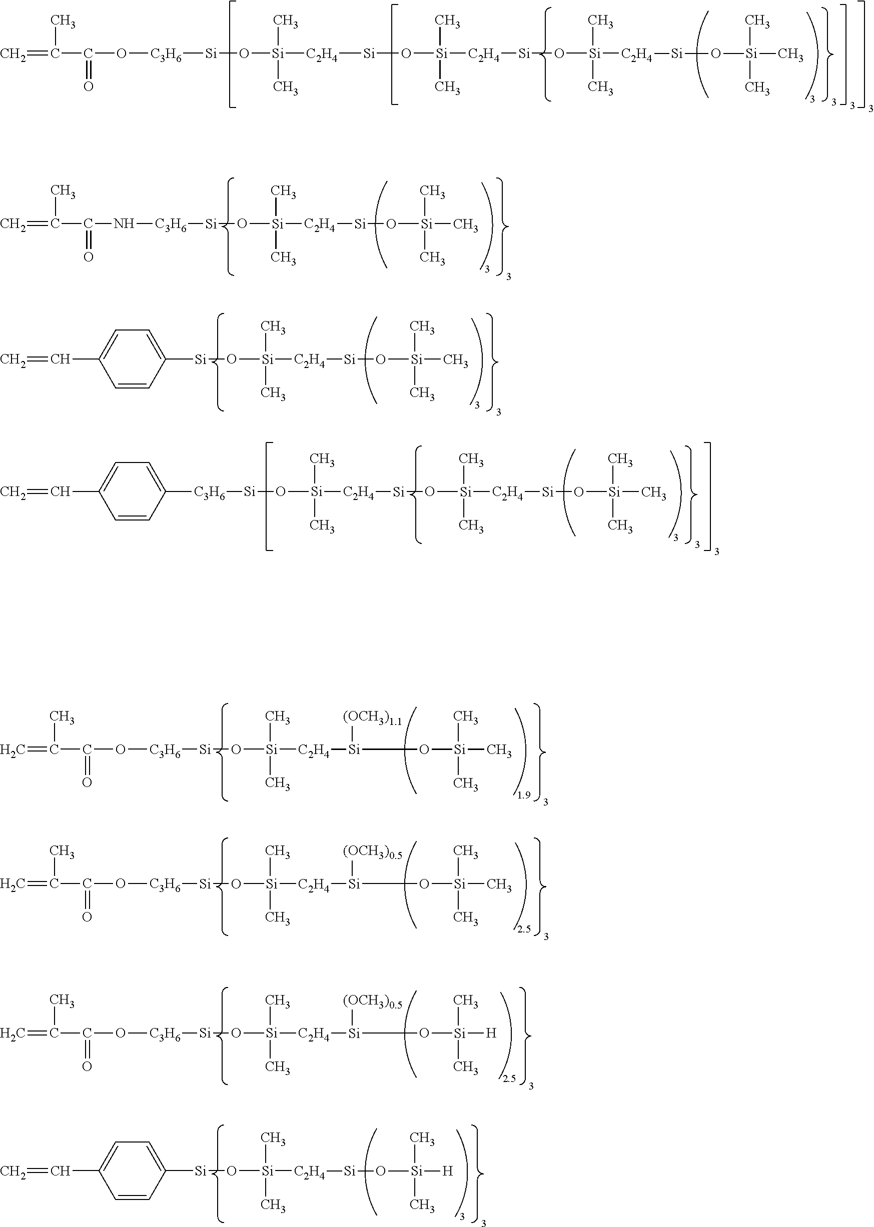

These carbosiloxane dendrimers can be produced using the production method for branched siloxane/silalkylene copolymers described in JP H11-001530 A (Appl. No. H09-171154). For example, one can be produced by conducting a hydrosilylation reaction on a silicon compound containing a silicon atom-bonded hydrogen atom represented by the following general formula

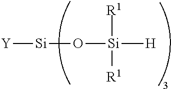

(where R¹ and Y are the same as above) and an alkenyl group-containing organosilicon compound. Examples of these silicon compounds that can be used include 3-methacryloxypropyltris (dimethylsiloxy) silane, 3-acryloxypropyltris (dimethylsiloxy) silane, and 4-vinylphenyltris (dimethylsiloxy) silane. Examples of these alkenyl group-containing organosilicon compounds that can be used include vinyl tris (trimethylsiloxy) silane, vinyl tris (dimethylphenylsiloxy) silane, and 5-hexenyltris (trimethylsiloxy) silane. The hydrosilylation reaction is preferably conducted in the presence of a transition metal catalyst such as chloroplatinic acid or a platinum vinyl siloxane complex.

Another preferred embodiment of monomer (A) used in the present invention is monomer (A2), which is represented by the following general formula (A-2).

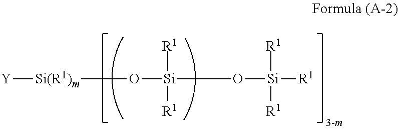

(In this formula, Y and R¹ are the same as above, m is 0, 1 or 2, and n is a number from 0 to 200 representing the average degree of polymerization.)

Specific examples of monomers represented by general formula (A-2) include the following compounds.

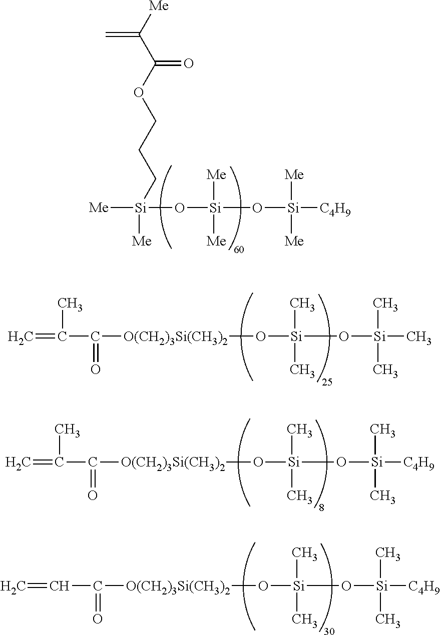

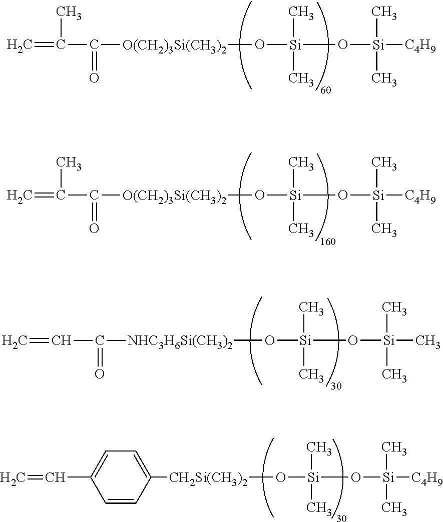

The following is an example of a compound in which m is 0 and n is 0 in general formula (A-2). It can be used as one embodiment of monomer (A) in the present invention.

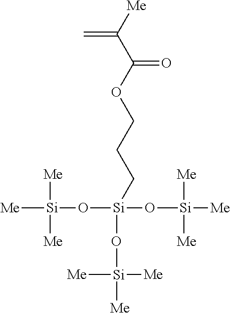

Another preferred embodiment of monomer (A) used in the present invention is monomer (A3), which is represented by the following general formula (A-3).

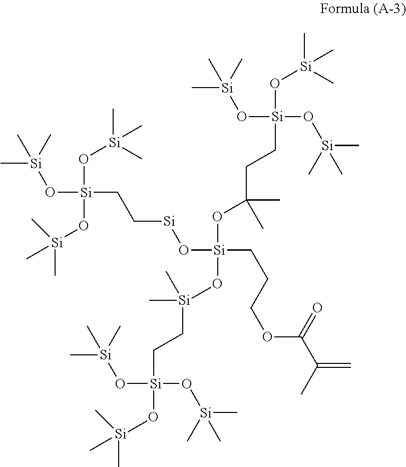

Another preferred embodiment of monomer (A) used in the present invention is monomer (A4), which is represented by the following general formula (A-4).

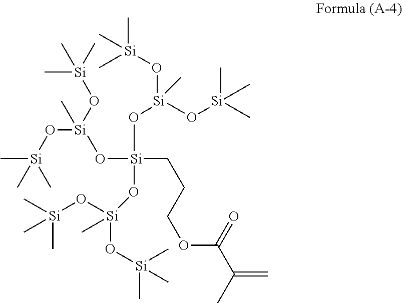

Monomers (A3) and (A4) are both branched organosilicons having an acrylate group as polymerizable group. Monomer (A3) has 16 silicon atoms. Monomer (A4) is structurally similar to monomer (A3), but has only 10 silicon atoms.

Another preferred embodiment of monomer (A) used in the present invention is monomer (A5), which is represented by the following general formula (A-5).

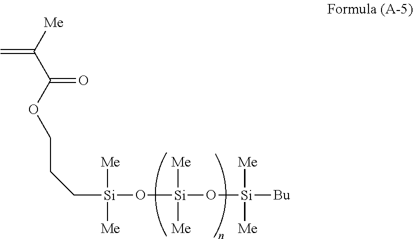

(In this formula, Me is methyl and Bu is butyl, n=0-120.)

Monomer (A5) is a linear organosilicon having an acrylate group as polymerizable group.

Another preferred embodiment of monomer (A) used in the present invention is monomer (A6), which is represented by the following general formula (A-6).

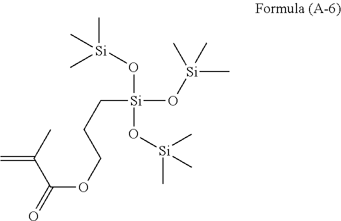

Like monomers (A3) and (A4), monomer (A6) is also a branched organosilicon having an acrylate group as polymerizable group, but it has only 4 silicon atoms.

Another preferred embodiment of monomer (A) used in the present invention is monomer (A7), which is represented by the following general formula (A-7).

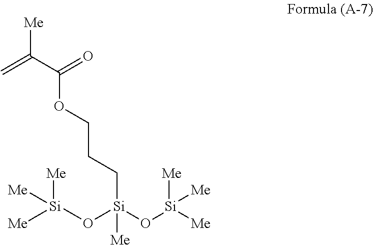

(In this formula, Me is methyl and Bu is butyl.)

Like monomer (A6), monomer (A7) is also a branched organosilicon having an acrylate group as polymerizable group, but it has only 3 silicon atoms.

The content of monomer (A) is 35% or more, and preferably 40% or more, and more preferably 45% or more, in terms of weight in the monomer composition. When at least this amount of monomer (A) is used in terms of weight, the water repellency and oil repellency of the resulting copolymer are higher and the water resistance and sebum resistance of a cosmetic using this copolymer are improved. Also, the content of monomer (A) is 70% or less, preferably 60% or less, and more preferably 55% or less, in terms of weight in the monomer composition.

### [Unsaturated Polymerizable Monomer (B)]

The unsaturated polymerizable monomer (B) is an unsaturated polymerizable monomer containing no or one silicon atom and having one polymerizable group in the molecule. But the type and properties of this monomer are not otherwise critical. The unsaturated polymerizable monomer (B) can be exemplified by one acidic group or salt thereof in the molecule, which is selected from the group consisting of: (meth)acrylic acid, crotonic acid, maleic acid, fumaric acid, itaconic acid, angelic acid, tigulinic acid, 2-carboxyethyl acrylate oligomers, styrene sulfonic acid, mono-[(2-hydroxyethyl) methacrylic acid] phosphate ester, mono-[(2-hydroxyethyl) acrylic acid] phosphate ester, ((2-hydroxyethyl) methacrylic acid) diphosphate ester, di [(2-hydroxyethyl) acrylic acid] phosphate ester, and salts thereof; lower alkyl (meth)acrylates or alkenyl (meth)acrylates such as methyl (meth)acrylate, ethyl (meth)acrylate, n-propyl (meth)acrylate, and isopropyl (meth)acrylate; higher (meth)acrylates such as n-butyl (meth)acrylate, isobutyl (meth)acrylate, tert-butyl (meth)acrylate, n-hexyl (meth)acrylate, cyclohexyl (meth)acrylate, 2-ethylhexyl (meth)acrylate, octyl (meth)acrylate, lauryl (meth)acrylate, cetyl (meth)acrylates, stearyl (meth)acrylate, isostearyl (meth)acrylate, and behenyl (meth)acrylate; lower fatty acid vinyl esters such as vinyl acetate and vinyl propionate; higher fatty acid esters such as vinyl butyrate, vinyl caproate, vinyl 2-ethylhexanoate, vinyl laurate, vinyl stearate, vinyl isostearate, and vinyl behenate; vinyl aromatic monomers such as styrene, vinyl toluene, phenyl (meth)acrylate, benzyl (meth)acrylate, phenoxyethyl (meth)acrylate, and vinyl pyrrolidone; amide group-containing vinyl monomers such as (meth)acrylamide, N-methylol (meth)acrylamide, N-methoxymethyl (meth)acrylamide, isobutoxymethoxy(meth)acrylamide, and N, N-dimethyl (meth)acrylamide; hydroxyl-containing vinyl monomers such as hydroxyethyl (meth)acrylate and hydroxypropyl alcohol (meth)acrylate; fluorinated vinyl monomers such as trifluoropropyl (meth)acrylate, perfluorobutylethyl (meth)acrylate, and perfluorooctylethyl (meth)acrylate; epoxy-functional vinyl monomers such as glycidyl (meth)acrylate, and 3,4-epoxycyclohexylmethyl (meth)acrylate; ether linkage containing vinyl ether bond-containing vinyl monomers such as tetrahydrofurfuryl (meth)acrylate, butoxyethyl (meth)acrylate, ethoxydiethylene glycol (meth)acrylate, polyethylene glycol (meth)acrylate, polypropylene glycol mono (meth)acrylate, polyethylene-polypropylene glycol (meth)acrylate, hydroxybutyl vinyl ether, cetyl vinyl ether, and 2-ethylhexyl vinyl ether; one silicon atom-containing vinyl monomers such as (meth)acryloxypropyl trimethoxysilane, (meth)acryloxypropyl triethoxysilane, (meth)acryloxyoctyl trimethoxysilane, (meth)acryloxyoctyl triethoxysilane, (meth)acryloxymethyl trimethoxysilane, (meth)acryloxymethyl triethoxysilane, trimethoxysilyl styrene, and triethoxysilyl styrene; styrene; butadiene; acrylonitrile; vinyl chloride; vinylidene chloride; (meth)acrylonitrile; dibutyl fumarate; maleic anhydride; (meth)acrylic glycidyl ether; quaternary ammonium salts derived from (meth)acrylic acid, such as 2-hydroxy-3-methacryloxypropyl trimethylammonium chloride, methacrylic acid esters of alcohols having a tertiary amine group such as methacrylic acid diethylamine ester, and quaternary ammonium salts of these.

Furthermore, an organic silicon compound having a vinyl-based radical polymerizable unsaturated group and a hydrolyzable group can also be used. In this case, the film strength becomes hard and water repellency durability is improved, which is preferable. Here, examples of the radically polymerizable group include a (meth)acryloxy group containing organic group, a (meth)acrylamide group-containing organic group, and a styryl group-containing organic group, as represented by the following general formulas, or an alkenyl group having from 2 to 10 carbon atoms, and a vinyloxy group and an allyloxy group.

Similarly, an unsaturated monomer having at least one acidic group or a salt thereof in a molecule can also be used. The unsaturated monomer having at least one acidic group or a salt thereof in a molecule is a compound having a radically polymerizable vinyl group and at least one acidic group or a salt thereof in a molecule. Examples of the acidic group include carboxylic acids, sulfonic acids, and phosphonic acids. Examples of salts thereof include alkali metal salts, alkaline earth metal salts, basic amino acid salts, ammonium salts, alkyl ammonium salts, alkyl amine salts, and alkanolamine salts, and specific examples include sodium salt, potassium salt, magnesium salt, calcium salt, L-arginine salt, L-histidine salt, L-lysine salt, ammonium salt, triethanolamine salt, aminomethyl propanediol salt, and complex salts thereof. Compounds having these acidic groups undergo a change in the hydrophilic-hydrophobic properties of the compound by releasing protons (H+) in an aqueous solution at respectively specific pH values or bonding with cationic components in the liquid to form a salt. Compounds with salts of acidic groups similarly: 65 undergo dissociation of the salt at a specific pH, and exhibit a change in the hydrophilic-hydrophobic properties of the compound. Therefore, by appropriately adding a compound having these acidic groups or salts thereof into a cosmetic material, an effect is achieved where washing away during cleaning is easy, even while also exhibiting favorable cosmetic retainability.

Similarly, for the purpose of improving water repellency or the like of the copolymer containing a carbosiloxane dendrimer structure in the present invention, an unsaturated monomer containing an organic group containing fluorine such as a perfluoroalkyl group and the like can also be used. An example is a vinyl-based monomer such as an acrylic monomer, a methacrylic monomer, or the like having an organic group containing fluorine such as a perfluoroalkyl group or the like.

### [Organosilicon-Functional Co-Polymer]

The copolymer containing a polysiloxane structure in the present invention is obtained by the copolymerizing of the aforementioned component (A) and component (B), and the mass ratio at the time of the copolymerization is preferably within a range of (A):(B)=35:65 to 70:30, more preferably 40:60 to 65:35, and even more preferably 40:60 to 60:40. In particular, the mass % of the component (A) described above is at least 35 mass %, and preferably at least 40 mass % relative to the total mass of component (A) and component (B), and component (A) is particularly preferably 40 mass % to 60 mass % of total monomer units.

The organosilicon-functional co-polymer of the present invention is obtained by conducting co-polymerization reaction of monomer (A) and monomer (B). Such organosilicon-functional co-polymer is a non-crosslinked co-polymer. Hence, unlike crosslinked co-polymers in prior arts that usually exhibit poor solubility due to their thermosetting property, the non-crosslinked organosilicon-functional co-polymer of the present invention can be more easily solved in various oils applicable to cosmetics.

The method used to copolymerize the copolymer for forming said solid particle of the present invention can be radical polymerization method, anionic polymerization method, cationic polymerization method, group transfer method, organometallic-mediated radical polymerization, or atom transfer radical addition method, but radical polymerization method is preferred. Radical polymerization can be conducted through at least one liquid phase polymerization reaction selected from solution polymerization, suspension polymerization, mini-emulsion polymerization and emulsion polymerization, but either solution polymerization or mini-emulsion polymerization is preferably used as the radical polymerization method, but solution polymerization is further preferably used as the radical polymerization method.

In mini-emulsion polymerization, a monomer composition consisting essentially of monomer (A) and monomer (B) is emulsified at first by emulsifier(s) and they are reacted in a lipid in the presence of a radical initiator at a temperature from 20 to 95° C. for 0.5 to 20 hours. Examples of emulsifiers that can be used in the mini-emulsion reaction include amphoteric surfactants, semipolar surfactants and reactive surfactants such as sodium lauryl sulfate, laureth-1 phosphate, polyglyceryl monostearate (polyglyceryl-10 stearate, a reaction product of polyglycerin having 10 glycerin repeating units and stearic acid), polyglyceryl monolaurate (polyglyceryl-10 laurate, a reaction product of polyglycerin having 10 glycerin repeating units and lauric acid), and the like; and high molecular weight emulsifiers such as an ethylene oxide 20 mol adduct of polyoxyethylene (C16) ether, an ethylene oxide 20 mol adduct of polyoxyethylene (C18) ether, and the like. As the radical initiator that can be used in the mini-emulsion reaction, as long as the radical polymerization initiator is a radical polymerization initiator that is typically used in emulsion polymerization of a vinyl polymer, it is not particularly limited. Examples thereof include water-soluble peroxides, including inorganic peroxides such as potassium persulfate, sodium persulfate, and ammonium persulfate; and organic peroxides such as t-butylperoxy maleic acid, succinic acid peroxide, and t-butyl hydroperoxide. When an oil-soluble radical initiator is used, the oil-soluble radical initiator may be mixed in and fed as a mixture comprising monomers (A) or/and monomer (B) before emusified, or the initiator is prerimilary emulsified and fed. For said mini-emulsion polymerization, it details have also been disclosed in US20190053999, which is incorporated herein by reference.

In solution polymerization, a monomer composition consisting essentially of monomer (A) and monomer (B) is reacted in the presence of a radical initiator in a solvent at a temperature from 50 to 150° C. for 3 to 20 hours. Examples of solvents that can be used in the polymerization reaction include aliphatic hydrocarbons such as hexane, octane, decane and cyclohexane; aromatic hydrocarbons such as benzene, toluene, and xylene; ethers such as diethyl ether, dibutyl ether, tetrahydrofuran, and dioxane; ketones such as acetone, methyl ethyl ketone, methyl isobutyl ketone, and diisobutyl ketone; esters such as methyl acetate, ethyl acetate, butyl acetate, and isobutyl acetate; alcohols such as methanol, ethanol, isopropyl alcohol, and butanol; and organosiloxane oligomers such as octamethylcyclotetrasiloxane, decamethylcyclopentasiloxane, hexamethyldisiloxane, and octamethyltrisiloxane.

Any radical initiator commonly used in the radical polymerization method can be used. Specific examples include azobis compounds such as 2,2′-azobis(isobutyronitrile), 2,2′-azobis(2-methylbutyronitrile), and 2,2′-azobis (2,4-dimethylvaleronitrile); and organic peroxides such as benzoyl peroxide, lauroyl peroxide, tert-butyl peroxybenzoate, tert-butylperoxy-2-ethylhexanoate, tert-hexylperoxy-2-ethylhexanoate, tert-Amyl peroxypivalate, tert-Butyl peroxypivalate, t-Hexyl peroxypivalate, Di(4-t-butylcyclohexyl) peroxydicarbonate, Di(3,5,5-trimethylhexanoyl) peroxide, and Diisopropyl peroxydicarbonate. These radical initiators can be used alone or on mixtures of two or more. The preferred amount depends on the target molecular weight of co-polymer. The amount of radical initiator used is preferably in a range from 0.005 to 10 parts by weight per 100 parts by weight monomer composition.

A chain transfer agent can also be added during polymerization in order to control molecular weight of the co-polymer. Specific examples of chain transfer agents include mercapto compounds such as 2-mercaptoethanol, butyl n-dodecyl mercaptan, 3-mercaptan, mercaptopropyltrimethoxysilane, polydimethylsiloxanes having a mercaptopropyl group, and mercaptopropionic acid; and halides such as methylene chloride, chloroform, carbon tetrachloride, butyl bromide, and 3-chloropropyltrimethoxysilane; and secondary alcohols such as Isopropyl alcohol and glycerin; and sulfurous acid (salt) such as sodium sodium sulfite; and sulfurous acid (salt); sodium bisulfite; sodium dithionite; potassium pyrosulfite; hydrogen peroxide, etc. Sulfurous acid (salt) such as sodium hydrogen sulfite; dithionic acid (salt) such as sodium dithionite; pyrosulfurous acid (salt) such as potassium pyrosulfite; and hydrogen peroxide.

After polymerization, purification can be performed by reducing the pressure under heating to remove the remaining unreacted vinyl monomers, by performing hydrogenation in the presence of a hydrogenation catalyst and in the presence or absence of a solvent to deodorize the product, and/or by contacting the product with nitrogen gas under reduced pressure to remove light substances. A purified product is especially preferred when used in external preparations which require low odor and compatibility with other cosmetic components. There are no particular restrictions on the solvents, reaction conditions, and reduced pressure conditions used in the hydrogenation reaction stripping process. Any solvent, reaction conditions, and reduced pressure conditions commonly used to purify organopolysiloxane copolymers can be selected.

The polymerization reaction product resulting from this process is then brought into contact with a nickel or palladium catalyst. By bringing the reaction product into contact with a palladium catalyst, the vinyl groups in the unreacted monomer remaining in the polymerization reaction product are saturated, and the irritation and odor of the product can be reduced before being added to cosmetics. Examples of palladium catalysts include, but are not limited to, palladium compounds such as tetrakis (triphenylphosphine) palladium (0) and dichlorobis (triphenylphosphine) palladium (II), as well as carbon-supported palladium, carbon-supported palladium hydroxide and platinum oxide. Carbon-supported palladium is the preferred catalyst. Palladium is a precious metal, this particular problem does not occur when a carbon-supported palladium catalyst is used as a heterogeneous catalyst. As a result, it is preferably used as the catalyst in the present invention.

The temperature at which the hydrogenation reaction product is brought into contact with the nickel catalyst or palladium catalyst is from 50 to 200° C., and preferably from 70 to 130° C. The pressure (absolute pressure) is from 1 to 1,000 kg/cm², and preferably from 2 to 100 kg/cm². The contact time is from 1 to 15 hours, and preferably 3 to 10 hours. The reaction can be performed in a solvent, and the solvent may be used directly during polymerization or solvent replacement may be performed. The solvent can be one of those mentioned above in relation to the polymerization reaction.

Stripping, reprecipitation, and filtration may also be performed during this process. Stripping, reprecipitation, filtration, pulverization, and/or classification can be performed after the hydrogenation reaction and following stripping and filtration.

The presence or absence of unreacted monomers in the resulting copolymer can be confirmed by the presence of a peak integrated value for ethylenically unsaturated groups (5.5 to 6.5 ppm) in ¹H-NMR. The end of the reaction can be confirmed by the disappearance or reduction in the peak derived from ethylenically unsaturated groups. More specifically, the comparison can be made using the ratio of the peak integrated value for ethylenically unsaturated groups to the product of the integrated value (0 to 0.3 ppm) for methyl groups derived from the unsaturated monomer having the polysiloxane structure and the weight percentage of the unsaturated monomer having the polysiloxane structure when added to the system (the residual unsaturation ratio). The residual unsaturation ratio for the copolymer is 0.1 or less, and preferably 0.02 or less.

### [Solid Particle]

After polymerization and followed drying process, obtained organosilicon-functional co-polymer is formed into solid particles having a long size of from 0.1 to 5,000 μm for its primary particles. Here, the method of such particlization is not restricted, and any production process known in the art can be used.

The method of producing the solid particle includes, for example, a method of pulverizing the organosilicon-functional co-polymer described above using a pulverizer, or a method of direct micronization in the presence of a solvent. The pulverizer may be, for example, but is not limited to, a roll mill, a ball mill, a jet mill, a turbo mill, or a planetary mill. Examples of a method of directly micronizing the organosilicon-functional co-polymer in the presence of a solvent include, spraying by a spray dryer, or micronizing using a biaxial kneader or a belt dryer.

In particular, by using a spray dryer or the like, solid particles having a regular spherical shape and an average primary particle diameter of from 0.1 to 5,000 μm. Furthermore, said solid particles obtained through the spray-dryer can be aggregated into particles having an average secondary particle diameter of from 0.5 to 5,000 μm, or 1.0 to 5,000 μm, or further preferably from 3.0 to 3,000 μm, or further more preferably from 5.0 to 2,000 μm. The heating and drying temperature of the spray dryer needs to be appropriately set based on the heat resistance of the organosilicon-functional co-polymer solid particles and the like. Note that in order to prevent secondary aggregation of the obtained solid particles, the temperature of the organosilicon-functional co-polymer solid particles is preferably controlled below the glass transition temperature thereof. The organosilicon-functional co-polymer solid particles thus obtained can be recovered by a cyclone, a bag filter, or the like.

Solvents may be used for the above particulation to the extent that they are not harmful to the desired properties of solid particle of the present invention. Examples of the solvents include, but are not limited to, aliphatic hydrocarbons such as n-hexane, cyclohexane, n-octane, n-decane, n-dodecane, methylcyclohexane, and n-heptane; aromatic hydrocarbons such as toluene, xylene, and mesitylene; ethers such as diethylether, diisopropylether, dibutykether, tetrahydrofuran and dipropyl ether; silicones such as hexamethyldisiloxane, octamethyltrisiloxane, and decamethyltetrasiloxane; esters such as esters such as methyl acetate, ethyl acetate, butyl acetate, and isobutyl acetate; and ketones such as acetone, methyl ethyl ketone, and methyl isobutyl ketone; alcohols such as methanol, ethanol, isopropyl alcohol, and butanol.

Obtained solid particle may have various shapes, as long as it can exhibit desired superior solubility and easy handling property as cosmetic ingredient at formulating into cosmetic compositions. In some embodiments, the shape of said solid particle is preferably selected from particle, powder, pellet, bead, short fiber, chopped strand, and crushed powder.

### Manufacturing Process of the Solvent-Soluble Solid Particle

In the present invention, a manufacturing process of the solvent-soluble solid particle described above is also provided. The manufacturing process comprises following steps:

Step (I): a step of preparing a solution or dispersion of organosilicon-functional co-polymer through polymerization reaction from a monomer composition consisting essentially of (A) an unsaturated polymerizable monomer having at least one organosilicon functionality and one polymerizable group in the molecule and (B) an unsaturated polymerizable monomer containing no or one silicon atom and having one polymerizable group in the molecule, wherein the mass ratio for said monomers (A) and (B) is in a range from 35:65 to 70:30 in said monomer composition; and

Step (II): a step of removing carrier fluid of water or solvent from the solution or dispersion of organosilicon-functional co-polymer prepared in above Step (I).

Step (I) is a step for preparing organosilicon-functional co-polymer through polymerization reaction. In some embodiments, said Step (I) is a step of preparing a solution or dispersion of organosilicon-functional co-polymer through at least one liquid phase polymerization reaction selected from solution polymerization, mini-emulsion polymerization and emulsion polymerization. Details of such polymerization reactions have been described in part [Organosilicon-functional co-polymer].

Step (II) is a step for preparing solid particle from organosilicon-functional co-polymer. In some embodiments, said Step (II) is a step of spray-drying process to obtain spherical particle of said organosilicon-functional co-polymer by removing carrier fluid of water or solvent from the solution or dispersion of organosilicon-functional co-polymer through spraying said solution or dispersion. Details of such spray-drying process have been described in part [Solid particle]. Also, said step (II) can be a step of removing carrier fluid of water or solvent to obtain solid organosilicon-functional co-polymer using stripping process. Specifically, this stripping process can be operated in barrel portion of one-axis or multi-axis screwed-extruder instrument. Through stripping process under reduced pressure and high temperature in said screwed-extruder instrument, solvent-removed organosilicon-functional co-polymer is kneaded with high shear and ejected from the outlet of said screwed-extruder instrument in heat-melting state. The melted organosilicon-functional co-polymer can be shaped into strand-form passing holes of strand-die equipped in the outlet of said screwed-extruder instrument. Optionally, this strand-shaped organosilicon-functional co-polymer is cooled using water-bath or other cooling device and following to cutting/pelletization process in below step (III).

In some embodiments, the manufacturing process of the solid particle described above further comprise below step:

Step (III): a step of forming the solid particle consisting essentially of organosilicon-functional co-polymer using at least one device selected from pelletizer, mill, pulverizer, grinder and drum flaker, following to said Step (II). As said extruder, pelletizer, mill, crusher, pulverizer, grinder, pastillator and drum flaker, any device known in the art can be used. At said step (III), for smooth-forming the solid particle and granulation, the viscosity or melting viscosity at processing temperature for said organosilicon-functional co-polymer is preferred to be within the range from 0.05 to 7,000 Pas. In particular, a pelletization process of organosilicon-functional co-polymer is one of preferred method to obtain the shaped solid particle of this organosilicon-functional co-polymer. That is to say, strand-shaped organosilicon-functional co-polymer using screwed-extruder instrument equipped with strand-die at its outlet is cutted by pelletizer into small-cut pellet of solid particle.

Step (III) is an optional step, but it is preferably to conduct step (III) so as to form the solid particle consisting essentially of organosilicon-functional co-polymer having desired long size of from 0.1 to 5,000 μm, or preferably from 0.5 to 5,000 μm, or more preferably, 1 to 5,000 μm.

Step (IV): a step of classifying the coarse solid particle consisting essentially of organosilicon-functional co-polymer using at least one device selected from with screen filter, mesh, punch plate, Cyclone and Dynamic air classifier.

Step (IV) is also an optional step, but it is preferably to conduct step (III) or (IV) so as to form the solid particle consisting essentially of organosilicon-functional co-polymer having desired long size of from 0.1 to 10,000 μm, or preferably from 0.1 to 5,000 μm, or more preferably, 1 to 5,000 μm.

### Use of the Solvent-Soluble Solid Particle

In the present invention, use of the solvent-soluble solid particle described above is also provided. Specifically, thanks for its high solubility in cosmetic solvents, the solvent-soluble solid particle of the present invention can be used as cosmetic ingredient, especially as cosmetic ingredient having film-forming function in human skin and/or hair. Especially, unlike current offering silicone acrylate that are mainly offered in dispersion form, the solvent-soluble solid particle of the present invention can be blended into a cosmetic directly. Of course, it is also possible to use it in conventional form of a composition dissolved in a solvent or dispersed in a dispersion medium.

### Cosmetic Composition and the Production Method Thereof

### [Cosmetic Composition]

In the present invention, a cosmetic composition comprising the solvent-soluble solid particle described above as well as the production method thereof are also provided. Specifically, the solvent-soluble solid particle of the present invention can be blended directly into cosmetic compositions, and is an extremely useful ingredient in cosmetics from the standpoint of handling and storage stability. Though potential cosmetic composition is various and not limited to, the solvent-soluble solid particle of the present invention can be used to replace co-polymer having organosilicon-functionality derived from aforementioned Si-functional monomer represented by (A-1) to (A-7), wherein carbosiloxane denrimer structure is preferred example of said organosilicon-functionality, in existing cosmetic formulations.

For example, the solvent-soluble solid particle of the present invention can partially or fully replace the ingredient of silicone acrylate co-polymer having carbosiloxane dendrimer structure (e.g. conventional product of FA 4001 CM Silicone Acrylate, FA 4002 ID Silicone Acrylate, FA 4003 silicone Acrylate, FA 4004 ID Silicone Acrylate, FA PEPS etc.) in cosmetic formulations in following patent publications;
WO2012/143344, WO2014/154701, WO2014/154700, WO2015/092632, WO2015/097110, WO2015/097103, WO2017/050699, WO2017/050922, WO2010/026538, WO2014/087183, WO2011/051323, JP2007-320960, WO2016/030842;
JP2010-018612, JP2011-016734, JP2011-016732, JP2011-016733, JP2011-016734, JP 2011-126807, JP 2011-126808, JP2013-001672, JP 2014-034568, JP 2014-040388, JP 2014-227358, JP2015-098451, JP 2015-137252, JP 2016-008200, JP2016-088848, JP 2016-121095, JP 2016-160191, JP 2018-090495;
JP2000-072784, JP07-309714, JP2007-320960, JP2014-040512, WO2017/061090, JP2011-149017, JP2014-040512, JP2014-040511, WO/2018/086139, WO/2018/186138, PCT/JP18/022412, PCT/JP18/022413.

Herein, the applicant clearly and intentionally teach and suggest the reader of this patent application to replace conventional co-polymer having organosilicon-functionality derived from aforementioned Si-functional monomer represented by (A-1) to (A-7) in conventional and available cosmetic formulations with the solvent-soluble solid particle of the present invention.

Furthermore, emulsion composition comprising the solvent-soluble solid particle of the present invention can be used to partially or fully replace silicone acrylate co-polymer emulsion in cosmetic formulations disclosed in WO2017/061090, WO/2018/086139, WO/2018/186138, PCT/JP18/022412, PCT/JP18/022413 and Research Disclosures: No.IPCOM000243971D, No.IPCOM0002457480.

By replacing existing silicone acrylate co-polymer having organosilicon-functionality derived from aforementioned Si-functional monomer represented by (A-1) to (A-7) with the solvent-soluble solid particle of the present invention in available and conventional cosmetic formulations, the skilled person in the art can anticipate and design similar or improved cosmetic formulations or compositions.

There are no particular restrictions on the amount blended into cosmetics, but the solvent-soluble solid particle of the present invention can be blended into a cosmetic composition in a range from 0.1 to 50 mass % of the entire cosmetic composition, preferably from 1 to 10 mass %. When the amount added is within this range, the properties of the solvent-soluble solid particle of the present invention can be imparted to a cosmetic composition, namely, film forming properties and film washability.

In addition to the solvent-soluble solid particle of the present invention, the cosmetic composition of the present invention can further comprise any conventional cosmetic ingredients, such as (D) an oil, (E) an alcohol, (F) a surfactant, (G) a powder or colorant, (H) a thickener or gelling agent, (I) an organically modified clay mineral, (J) a silicone resin, (K) a silicone gum, (L) a silicone elastomer, (M) an organically modified silicone, (N) a UV protection component, (O) a water-soluble polymer, and water.

### (D) Oil

The oil can be any animal oil, vegetable oil, or mineral oil commonly used in cosmetics. The oil can be solid, semi-solid or liquid and can be non-volatile, semi-volatile or volatile. An oil is used to impart lubrication to skin and hair, and to make skin soft and impart a moist feeling. An oil can also be used to dilute the organosilicon-functional co-polymer of the present invention to obtain a copolymer composition. The oil is preferably at least one type selected from among (D1) a silicone oil and (D2) an organic oil that is a liquid at a temperature from 5 to 100° C. The type and viscosity of the oil depends on the type of cosmetic and the intended use. These oils are blended into a cosmetic composition of the present invention at the same time as the composition.

### (D1) Silicone Oils

The silicone-based oils are generally hydrophobic and their molecular structures may be cyclic, linear or branched. Here, the molecular structure may be cyclic, linear or branched. The viscosity of the silicone-based oil at 25° C. is usually in a range from 0.65 to 100,000 mm²/s and preferably in a range from 0.65 to 10,000 mm²/s. The silicone oil agent may have volatility and this is preferred.

Examples of silicone-based oils include cyclic organopolysiloxanes, linear organopolysiloxanes and branched organopolysiloxanes. Among these, volatile cyclic organopolysiloxanes, linear organopolysiloxanes and branched organopolysiloxanes are preferred.

The silicone oil can be an organopolysiloxane represented by General Formula (3), (4) or (5) below.

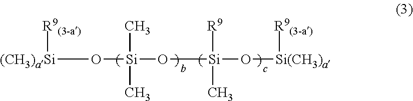

(In this formula, R⁹ is a hydrogen atom or a group selected from among a hydroxyl group, a monovalent unsubstituted or fluorine- or amino-substituted alkyl group, aryl group and alkoxy group having from 1 to 30 carbon atoms, and (CH₃)₃SiO{(CH₃)₂SiO}_{I}Si(CH₃)₂CH₂CH₂— (where I is an integer from 0 to 1,000), a′ is an integer from 0 to 3, b is an integer from 0 to 1,000, and c is an integer from 0 to 1000, provided 1≤b+c≤2,000.)

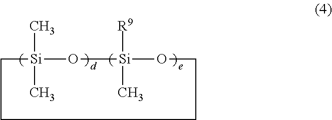

(In this formula, R⁹ is the same as above, d is an integer from 0 to 8, and e is an integer from 0 to 8, provided 3≤d+e≤8.)

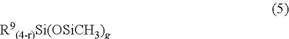

(In this formula, R⁹ is the same as above, f is an integer from 1 to 4, and g is an integer from 0 to 500.)

Examples of monovalent unsubstituted or fluorine- or amino-substituted alkyl groups, aryl groups and alkoxy groups having from 1 to 30 carbon atoms include linear or branched alkyl groups having from 1 to 30 carbon atoms such as a methyl group, an ethyl group, a propyl group, a butyl group, a pentyl group, a hexyl group, a heptyl group, an octyl group, a decyl group, and a dodecyl group; cycloalkyl groups having from 3 to 30 carbon atoms such as a cyclopentyl group and a cyclohexyl group; aryl groups having from 6 to 30 carbon atoms such as a phenyl group, a tolyl group, a xylyl group, and a naphthyl group; alkoxy groups having 1 to 30 carbon atoms such as a methoxy group, an ethoxy group, and a propoxy group; and groups in which hydrogen atoms bonded to carbon atoms in any of these groups have been at least partially replaced by a fluorine atom or an amino group. An unsubstituted alkyl group or aryl group is preferred, an unsubstituted alkyl group or aryl group having from 1 to 6 carbon atoms is more preferred, and a methyl group, ethyl group or phenyl group is especially preferred.

Examples of silicone oils having these structures include cyclic organopolysiloxanes. Specific examples include hexamethylcyclotrisiloxane, decamethylcyclopentasiloxane, octamethylcyclotetrasiloxane, dodecamethylcyclohexasiloxane, 1,1-ethylhexamethylcyclotetrasiloxane, phenylheptamethylcyclotetrasiloxane, 1,1-diphenylhexamethylcyclotetrasiloxane, 1,3,5,7-tetravinyltetramethylcyclotetrasiloxane, 1,3,5,7-tetramethylcyclotetrasiloxane, 1,3,5,7-tetracyclohexyltetramethylcyclotetrasiloxane, tris (3,3,3-trifluoropropyl)trimethylcyclotrisiloxane, 1,3,5,7-tetra (3-methacryloxypropyl) tetramethylcyclotetrasiloxane, 1,3,5,7-tetra (3-acryloxypropyl) tetramethylcyclotetrasiloxane, 1,3,5,7-tetra (3-carboxypropyl) tetramethylcyclotetrasiloxane, 1,3,5,7-tetra (3-vinyloxypropyl) tetramethylcyclotetrasiloxane, 1,3,5,7-tetra (p-vinylphenyl) tetramethylcyclotetrasiloxane, 1,3,5,7-tetra [3-(p-vinylphenyl) propyl] tetramethylcyclotetrasiloxane, 1,3,5,7-tetra (N-acryloyl-N-methyl-3-aminopropyl) tetramethylcyclotetrasiloxane, and 1,3,5,7-tetra (N, N-bis (lauroyl)-3-aminopropyl) tetramethylcyclotetrasiloxane.

Examples of linear organopolysiloxanes include dimethylpolysiloxane capped at both ends of the molecular chain with a trimethylsiloxy group (dimethyl silicone with a low viscosity of 2 mPa·s or 6 mPa·s to dimethyl silicone with a high viscosity of 1 million mPa·s), diethylpolysiloxane capped at both ends of the molecular chain with a triethylsiloxy group, organohydrogenpolysiloxane, methylphenylpolysiloxane capped at both ends of molecular chain with a trimethylsiloxy group, the dimethylsiloxane/methylphenylsiloxane copolymers capped at both ends of the molecular chain with a trimethylsiloxy group, diphenyl polysiloxane capped at both ends of the molecular chain with a trimethylsiloxy group, dimethylsiloxane/diphenylsiloxane copolymers capped at both ends of the molecular chain with a trimethylsiloxy group, dimethylsiloxane/methylphenylsiloxane copolymers capped at both ends of the molecular chain with a trimethylsiloxy group, diphenyl polysiloxane capped at both ends of the molecular chain with a trimethylsiloxy group, dimethylsiloxane/diphenylsiloxane copolymers capped at both ends of the molecular chain with a trimethylsiloxy group, diphenylpolysiloxane capped at both ends of the molecular chain with a trimethylsiloxy group, dimethylsiloxane/diphenylsiloxane copolymer capped at both ends of the molecular chain with a trimethylsiloxy group, trimethylpentaphenyl trisiloxane, phenyl (trimethylsiloxy) siloxane, methyl alkyl polysiloxane capped at both ends of the molecular chain with trimethylsiloxy group, dimethylpolysiloxane/methylalkylsiloxane copolymer capped at both ends of the molecular chain with a trimethylsiloxy group, dimethylsiloxane-methyl (3,3,3-trifluoropropyl) siloxane copolymer capped at both ends of the molecular chain with a trimethylsiloxy group, α,ω-dihydroxypolydimethylsiloxane, α,ω-diethoxypolydimethylsiloxane, 1,1,1,3,5,5,5-heptamethyl-3-octyltrisiloxane, 1,1,1,3,5,5,5-heptamethyl-3-dodecyltrisiloxane, 1,1,1,3,5,5,5-heptamethyl-3-hexadecyltrisiloxane, tristrimethylsiloxymethylsilane, tristrimethylsiloxyalkylsilane, tetrakistrimethylsiloxysilane, tetramethyl-1,3-dihydroxydisiloxane, octamethyl-1,7-dihydroxytetrasiloxane, hexamethyl-1,5-diethoxytrisiloxane, hexamethyldisiloxane, octamethyltrisiloxane, higher alkoxy-modified silicones, and higher fatty acid modified silicones.

Examples of branched organopolysiloxanes include methyl tristrimethylsiloxysilane, ethyl tristrimethylsiloxysilane, propyl tristrimethylsiloxysilane, tetrakis trimethylsiloxysilane, and phenyl tristrimethylsiloxysilane.

When a cosmetic or composition of the present invention containing at least one of these silicone-based oils is used as an ingredient in a cosmetic composition, aging stability can be improved and the smooth feel characteristic of silicone oil can be realized. Among these silicone-based oils, decamethylcyclopentasiloxane a linear organopolysiloxane with a viscosity in the low viscosity range from 2 to 6 mPa·s, 1,1,1,3,5,5,5-heptamethyl-3-octyltrisiloxane (caprylyl methicone), and tris trimethylsiloxymethylsilane (M3T) are especially preferred.

### (D2) Organic Oils

Examples of organic oils include (D2-1) hydrocarbon oils, (D2-2) fatty acid ester oils, higher alcohols, higher fatty acids, oils and fats, and fluorinated oils, and (D2-3) light ester oil. There are no particular restrictions in the present invention, but the organic oil is preferably a liquid at a temperature from 5 to 100° C. Also, hydrocarbon oils and/or fatty acid ester oils are preferred. These can be used alone or in combination with other organic oils and/or silicone-based oils. When the appropriate oils are combined, the stability of the composition and/or cosmetic over time is improved, and the feel required of each cosmetic can be imparted. The smooth feel characteristic of silicone oil can be imparted by blending in a silicone-based oil, a refreshing feel can be imparted to the skin by blending in a highly volatile oil, and a smooth feeling and moisturizing effect (moist feeling) can be imparted to the skin and hair by using a hydrocarbon oil and/or fatty acid ester oil in combination with the silicone-based oil.

Examples of hydrocarbon oils (D2-1) include liquid paraffin, light liquid isoparaffin, heavy liquid isoparaffin, vaseline, n-paraffin, isoparaffin, isododecane, isohexadecane, polyisobutylene, hydrogenated polyisobutylene, polybutene, ozokerite, ceresin, microcrystalline wax, paraffin wax, polyethylene wax, polyethylene/polypropylene wax, squalane, squalene, pristane, and polyisoprene. Here, linear alkane from vegetable origin can be used and volatile linear alkane understood 9-17C, preferably 11-13C are used. Use of isododecane, undecane and/or tridecane is especially preferred in a cosmetic composition of the present invention because it has excellent volatility, excellent compatibility and affinity (combination stability) with other cosmetic ingredients, and imparts a refreshing feel to the skin.

Examples of fatty acid ester oils (D2-2) include hexyldecyl octoate, cetyl octanoate, isopropyl myristate, isopropyl palmitate, butyl stearate, hexyl laurate, myristyl myristate, oleyl oleate, decyl oleate, octyldodecyl myristate, hexyldecyl dimethyloctanoate, cetyl lactate, myristyl lactate, diethyl phthalate, dibutyl phthalate, lanolin acetate, ethylene glycol monostearate, propylene glycol monostearate, propylene glycol dioleate, glyceryl monostearate, glyceryl monooleate, glyceryl tri-2-ethylhexanoate, trimethylolpropane tri-2-ethylhexanoate, ditrimethylolpropane triethylhexanoate, (isostearic acid/sebacic acid) ditrimethylolpropane, trimethylolpropane trioctanoate, trimethylolpropane triisostearate, diisopropyl adipate, diisobutyl adipate, 2-hexyldecyl adipate, di-2-heptylundecyl adipate, diisostearyl malate, hydrogenated castor oil monoisostearate, N-alkyl glycol monoisostearate, octyldodecyl isostearate, isopropyl isostearate, isocetyl isostearate, ethylene glycol di-2-ethylhexanoate, cetyl 2-ethylhexanoate, pentaerythritol tetra-2-ethylhexanoate, octyldodecyl gum ester, ethyl oleate, octyldodecyl oleate, neopentyl glycol dicaprate, triethyl citrate, 2-ethylhexyl succinate, dioctyl succinate, isocetyl stearate, diisopropyl sebacate, di-2-ethylhexyl sebacate, diethyl sebacate, dioctyl sebacate, dibutyl octyl sebacate, cetyl palmitate, octyldodecyl palmitate, octyl palmitate, 2-ethylhexyl palmitate, 2-hexyldecyl palmitate, 2-heptylundecyl palmitate, cholesteryl 12-hydroxystearylate, dipentaerythritol fatty acid ester, 2-hexyldecyl myristate, ethyl laurate, N-lauroyl-L-glutamic acid-2-octyldodecyl ester, di (cholesteryl/behenyl/octyldodecyl)N-lauroyl-L-glutamate, di (cholesteryl/octyldodecyl)N-lauroyl-L-glutamate, di (phytosteryl/behenyl/octyldodecyl)N-lauroyl-L-glutamate, di (phytosteryl/octyldodecyl)N-lauroyl-L-glutamate, isopropyl N-lauroyl sarcosine, diisostearyl malate, neopentyl glycol dioctanoate, isodecyl neopentanoate, isotridecyl neopentanoate, isostearyl neopentanoate, isononyl isononanoate, isotridecyl isononanoate, octyl isononanoate, isotridecyl isononanoate, diethyl pentanediol dineopentanoate, methyl neopentanoate pentanediol, octyldodecyl neodecanoate, 2-butyl-2-ethyl dioctanoate-1,3-propanediol, pentaerythrityl tetraoctanoate, hydrogenated rosin pentaerythrityl, pentaerythrityl triethylhexanoate, dipentaerythrityl (hydroxystearate/stearate/rosinate), polyglyceryl tetraisostearate, polyglyceryl-10 nonisostearate, polyglyceryl deca (erucate/isostearate/ricinoleate)-8, diglyceryl (hexyldecanoate/sebacate) oligoester, glycol distearate (ethylene glycol distearate), diisopropyl dimer dilinoleate, diisostearyl dimer dilinoleate, dimer (isostearyl/phytosteryl) dilinoleate, dimer (phytosteryl/behenyl) dilinoleate, dimer (phytosteryl/isostearyl/cetyl/stearyl/behenyl) dilinoleate, dimer dilinoleate dimer dilinoleate, dimer dilinoleyl diisostearate, dimer linoleyl hydrogenated rosin condensate, dimer dilinoleate hydrogenated castor oil, hydroxyalkyl dimer dilinoleyl ether, glyceryl triisooctanoate, glyceryl triisostearate, glyceryl trimyristate, glyceryl triisopalmitate, glyceryl trioctanoate, glyceryl trioleate, glyceryl diisostearate, glyceryl tri (caprylate/caprate), glyceryl tri (caprylate/caprate/myristate/stearate), hydrogenated rosin triglyceride (hydrogenated ester gum), rosin triglyceride (ester gum), glyceryl beiconate eicosanedioate, glyceryl di-2-heptylundecanoate, diglyceryl myristate isostearate, cholesteryl acetate, cholesteryl nonanoate, cholesteryl stearate, cholesteryl isostearate, cholesteryl oleate, cholesteryl 12-hydroxystearate, cholesteryl macadamia oil fatty acid, phytosteryl macadamia nut oil fatty acid, phytosteryl isostearate, cholesteryl soft lanolin fatty acid, cholesteryl hard lanolin fatty acid, cholesteryl long-chain branched fatty acid, cholesteryl long-chain a-hydroxy fatty acid, octyldodecyl ricinoleate, octyldodecyl lanolin fatty acid, octyldodecyl erucate, isostearic acid hydrogenated castor oil, ethyl avocado oil fatty acid, and isopropyl lanolin fatty acid. Lanolin and lanolin derivatives can also be used as fatty acid ester oils.

In addition to ones mentioned above, oils and fats, higher alcohols, higher fatty acids, and fluorine-based oils may be used as an oil, or two or more of these may be used in combination. For example, two or more of the oils listed below may be used in combination. The following are specific examples of additional oils that can be used in the present invention. One or more selected from among these oils and fats, higher alcohols, higher fatty acids, and fluorine-based oils can be used.

Among oils and fats, natural animal and vegetable fats and oils and semi-synthetic fats and oils that can be used include avocado oil, linseed oil, almond oil, Chinese insect wax, eno oil, olive oil, cocoa butter, kapok low, kaya oil, carnauba wax, liver oil, candelilla wax, beef tallow, beef leg fat, beef bone fat, hardened tallow, apricot kernel oil, whale wax, hardened oil, wheat germ oil, sesame oil, rice germ oil, rice bran oil, sugar cane wax, sasanqua oil, safflower oil, shea butter, Chinese tung oil, cinnamon oil, jojoba wax, olive squalane, shellac wax, turtle oil, soybean oil, teaseed oil, camellia oil, evening primrose oil, corn oil, pig lard, rapeseed oil, Japanese tung oil, rice bran wax, germ oil, horse fat, persic oil, palm oil, palm kernel oil, castor oil, hydrogenated castor oil, castor oil fatty acid methyl ester, sunflower oil, grape oil, bayberry wax, jojoba oil, hydrogenated jojoba ester, macadamia nut oil, beeswax, mink oil, cottonseed oil, cotton wax, Japan wax, Japan wax kernel oil, montan wax, coconut oil, hardened coconut oil, tri-coconut oil fatty acid glyceride, sheep fat, peanut oil, lanolin, liquid lanolin, reduced lanolin, lanolin alcohol, hard lanolin, lanolin acetate, lanolin fatty acid isopropyl, POE lanolin alcohol ether, POE lanolin alcohol acetate, lanolin fatty acid polyethylene glycol, POE hydrogenated lanolin alcohol ether, and egg yolk oil. Here, POE refers to polyoxyethylene.

A higher alcohol has from 10 to 30 carbon atoms. A higher alcohol is a saturated or unsaturated monohydric aliphatic alcohol. Some of the hydrocarbon groups may be linear or branched, but linear groups are preferred. Examples of higher alcohols having from 10 to 30 carbon atoms include lauryl alcohol, myristyl alcohol, palmityl alcohol, stearyl alcohol, behenyl alcohol, hexadecyl alcohol, oleyl alcohol, isostearyl alcohol, hexyldecanol, octyldodecanol, cetostearyl alcohol, 2-decyltetradecinol, cholesterol, sitosterol, phytosterol, lanosterol, lanolin alcohol, hydrogenated lanolin alcohol, POE cholesterol ether, monostearyl glycerin ether (batyl alcohol), and monooleyl glyceryl ether (selachyl alcohol). Preferably, in the present invention, a higher alcohol having a melting point of 40 to 80° C. is used alone or a combination of higher alcohols having a melting point of 40 to 70° C. is used. These higher alcohols, together with a surfactant, form an aggregate called an a-gel, and have the function of increasing the viscosity of the preparation and stabilizing the emulsion. As a result, they are especially useful as a base in a cosmetic emulsion.

Examples of higher fatty acids include lauric acid, myristic acid, palmitic acid, stearic acid, behenic acid, undecylenic acid, oleic acid, linoleic acid, linolenic acid, arachidonic acid, eicosapentaenoic acid (EPA), docosahexaenoic acid (DHA), isostearic acid, and 12-hydroxystearic acid.

Examples of fluorine-based oils include perfluoropolyether, perfluorodecalin, and perfluorooctane.

Examples of light ester oils (D2-3) include methyl formate, ethyl formate, n-propyl formate, i-propyl formate, n-butyl formate, i-butyl formate, s-butyl formate, t-butyl formate, methyl acetate, ethyl acetate, n-propyl acetate, i-propyl acetate, n-butyl acetate, i-butyl acetate, s-butyl acetate, t-butyl acetate, methyl propionate, ethyl propionate, n-propyl propionate, i-propyl propionate, n-butyl propionate, i-butyl propionate, s-butyl propionate, t-butyl propionate, methyl butyrate, ethyl butyrate, n-propyl butyrate, i-propyl butyrate, n-butyl butyrate, i-butyl butyrate, s-butyl butyrate, t-butyl butyrate.

### (E) Alcohols

Organosilicon-functional co-polymer of the present invention may be used after being dispersed or dissolved in an alcohol. Because organosilicon-functional co-polymer of the present invention have excellent affinity with alcohols, which are commonly used as a component in cosmetics, alcohols can also be used in a cosmetic formulation. One or more polyhydric alcohols and/or lower monohydric alcohols can be used. Examples of lower alcohols include ethanol, isopropanol, n-propanol, t-butanol, and sec-butanol. Ethanol is preferred. Examples of polyhydric alcohols include dihydric alcohols such as 1,3-propanediol, 1,3-butylene glycol, 1,2-butylene glycol, propylene glycol, trimethylene glycol, tetramethylene glycol, 2,3-butylene glycol, pentamethylene glycol, 2-butene-1,4-diol, dibutylene glycol, pentyl glycol, hexylene glycol, and octylene glycol; trihydric alcohols such as glycerin, trimethylolpropane, and 1,2,6-hexanetriol; tetrahydric alcohols and higher such as pentaerythritol and xylitol; and sugar alcohols such as sorbitol, mannitol, maltitol, maltotriose, sucrose, erythritol, glucose, fructose, starch degradation products, maltose, xylitolose, and starch degraded sugar reduced alcohols. Examples other than these lower polyhydric alcohols include polyhydric alcohol polymers such as diethylene glycol, dipropylene glycol, triethylene glycol, polypropylene glycol, tetraethylene glycol, diglycerin, polyethylene glycol, triglycerin, tetraglycerin, and polyglycerin. Among these, ethanol, 1,3-propanediol, 1,3-butylene glycol, sorbitol, dipropylene glycol, glycerin, and polyethylene glycol are especially preferred.

(F) surfactants

A cosmetic composition containing the solvent-soluble solid particle of the present invention can include (F) a surfactant as an optional component. Depending on the intended use, the (F) surfactant can be one or more surfactants selected from a group consisting of (F1) a silicone-based surfactant, (F2) an anionic surfactant, (F3) a cationic surfactant, (F4) a nonionic surfactant, (F5) an amphoteric surfactant, and (F6) a semipolar surfactant. For each, reactive surfactant having polymerizable unsaturated group is also applicable.

Examples of (F1) silicone-based surfactants include polyglyceryl-modified silicones, diglyceryl-modified silicones, glyceryl-modified silicones, sugar-modified silicones, fluorinated polyether-modified silicones, polyether-modified silicones, carboxylic acid-modified silicones, linear silicone/polyether block copolymers (polysilicone-13, etc.), long-chain alkyl/polyether co-modified silicones, polyglyceryl-modified silicone elastomers, diglyceryl knitted elastomers, glyceryl-modified elastomers, and polyether modified elastomers. The silicones and elastomers described above provided with an alkyl branch, a linear silicone branch or a siloxane dendrimer branch at the same time as the hydrophilic group, if necessary, can also be used. Commercially available products include SH 3771 M, SH 3772 M, SH 3773 M, SH 3775 M, BY 22-008 M, BY 11-030, ES-5226 DM Formulation Aid, ES-5227 DM Formulation Aid, ES-5373 FORMULATION AID, ES-5612 FORMULATION AID, ES-5300 FORMULATION AID, ES-5600 SILICONE GLYCEROL EMULSIFIER, ES-5700 FORMULATION AID, and ES-5800 FORMULATION AID (all from Dow Toray).

Examples of (F2) anionic surfactants include saturated or unsaturated fatty acid salts (such as sodium laurate, sodium stearate, sodium oleate, and sodium linolenate), alkyl sulfates, alkylbenzenesulfonic acids (such as hexylbenzenesulfonic acid, octylbenzenesulfonic acid, and dodecylbenzenesulfonic acid) and salts thereof, polyoxyalkylene alkyl ether sulfates, polyoxyalkylene alkenyl ether sulfates, polyoxyethylene alkyl sulfates, alkyl sulfosuccinates, polyoxyalkylene sulfosuccinate alkyl ester salts, polyoxyalkylene alkyl phenyl ether sulfates, alkane sulfonates, octyltrimethylammonium hydroxide, dodecyltrimethylammonium hydroxide, alkyl sulfonates, polyoxyethylene alkyl phenyl ether sulfates, polyoxyalkylene alkyl ether acetates, alkyl phosphates, polyoxyalkylene alkyl ether phosphate, acyl glutamate, α-acyl sulfonates, alkyl sulfonates, alkyl allyl sulfonates, α-olefin sulfonate, alkyl naphthalene sulfonates, alkane sulfonates, alkyl or alkenyl sulfates, alkyl amide sulfate, alkyl or alkenyl phosphates, alkylamidophosphates, alkyloylalkyl taurine salts, N-acyl amino acid salts, sulfosuccinate, alkyl ether carboxylate, amide ether carboxylate, α-sulfo fatty acid ester salt, alanine derivatives, glycine derivatives, and arginine derivatives. Salts include alkali metal salts such as sodium salts, alkaline earth metal salts such as magnesium salts, alkanolamine salts such as triethanolamine salts, and ammonium salts.

Examples of (F3) cationic surfactants include alkyl trimethyl ammonium chloride, stearyl trimethyl ammonium chloride, lauryl trimethyl ammonium chloride, cetyl trimethyl ammonium chloride, alkyl trimethyl ammonium chloride tallow, behenyl trimethyl ammonium chloride, stearyl trimethyl ammonium bromide, behenyl trimethyl ammonium bromide, distearyl dimethyl ammonium chloride, dicocoyl dimethyl ammonium chloride, dioctyl dimethyl ammonium chloride, di (POE) oleyl methyl ammonium chloride (2EO), benzalkonium chloride, alkyl benzalkonium chloride, alkyl dimethyl benzalkonium chloride, benzethonium chloride, stearyl dimethyl benzyl ammonium chloride, lanolin-derived quaternary ammonium salts, diethyl aminoethylamide stearate, dimethyl aminopropylamide stearate, amidopropyl dimethyl hydroxypropyl ammonium behenate chloride, stearoylcholaminoformyl methyl pyridinium chloride, cetylpyridinium chloride, tall oil alkylbenzylhydroxyethyl imidazolinium chloride, and benzyl ammonium salt.

Examples of (F4) nonionic surfactants include polyglyceryl diisostearate or diglyceryl polyhydroxystearate, isostearyl glyceryl ether, polyoxyalkylene ethers, polyoxyalkylene alkyl ethers, polyoxyalkylene fatty acid esters, polyoxyalkylene fatty acid diesters, polyoxyalkylene resin acid esters, polyoxyalkylene (hardened) castor oils, polyoxyalkylene alkylphenols, polyoxyalkylene alkyl phenyl ethers, polyoxyalkylene phenyl ethers, polyoxyalkylene alkyl esters, polyoxyalkylene alkyl esters, sorbitan fatty acid esters, polyoxyalkylene sorbitan alkyl esters, polyoxyalkylene sorbitan fatty acid esters, polyoxyalkylene sorbite fatty acid esters, polyoxyalkylene glycerin fatty acid esters, polyglycerin alkyl ethers, polyglycerin fatty acid esters, sucrose fatty acid esters, fatty acid alkanolamides, alkyl glucosides, polyoxyalkylene fatty acid bisphenyl ethers, polypropylene glycol, diethylene glycol, polyoxyethylene/polyoxypropylene block polymers, alkyl polyoxyethylene/polyoxypropylene block polymer ethers, block polyoxyethylene/polyoxypropylene polymers, alkyl polyoxyethylene/polyoxypropylene block polymer ethers, and fluorine-based surfactants.

Examples of (F5) amphoteric surfactants include imidazoline-type, amidobetaine-type, alkylbetaine-type, alkylamidobetaine-type, alkylsulfobetaine-type, amidesulfobetaine-type, hydroxysulfobetaine-type, carbobetaine-type, phosphobetaine-type, aminocarboxylic acid-type, and amidoamino acid-type amphoteric surfactants. Specific examples include such as 2-undecyl-N,N,N-imidazoline-type amphoteric surfactants (hydroxyethyl carboxymethyl)-2-imidazoline sodium and 2-cocoyl-2-imitazolinium hydroxide-1-carboxyethyloxy disodium salts; alkyl betaine-type amphoteric surfactants such as betaine lauryl dimethylaminoacetate and myristyl betaine; amidobetaine-type amphoteric surfactants such as coconut oil fatty acid amidopropyl dimethylaminoacetic acid betaine, palm kernel oil fatty acid amidopropyl dimethylaminoacetic acid betaine, beef tallow fatty acid amidopropyl dimethylaminoacetic acid betaine, hardened tallow fatty acid amidopropyl dimethylaminoacetic acid betaine, betaine laurate amidopropyl dimethylaminoacetate, betaine myristic acid amidopropyl dimethylaminoacetate, betaine amidopropyl dimethylaminoacetate palmitate, betaineamide dimethyl dimethylaminoacetate acetate, and amidopropyl oleate dimethylaminoacetic acid betaine; alkylsulfobetaine-type amphoteric surfactants such as coconut oil fatty acid dimethylsulfopropylbetaine; alkylhydroxysulfobetaine-type amphoteric surfactants such as lauryldimethyl aminohydroxysulfobetaine; and amidoamino acid-type amphoteric surfactants such as sodium N-lauroyl-N′-hydroxyethyl-N′-carboxymethylethylenediamine, sodium N-oleoyl-N′-hydroxyethyl-N′-carboxymethylethylenediamine, sodium N-cocoyl-N′-hydroxyethyl-N′-carboxymethylethylenediamine, potassium N-lauroyl-N′-hydroxyethyl-N′-carboxymethylethylenediamine, potassium N-oleoyl-N′-hydroxyethyl-N′-carboxymethylethylenediamine, sodium N-lauroyl-N-hydroxyethyl-N′-carboxymethylethylenediamine, sodium N-oleoyl-N-hydroxyethyl-N′-carboxymethylethylenediamine, sodium N-cocoyl-N-hydroxyethyl-N′-carboxymethylethylenediamine, monosodium N-lauroyl-N-hydroxyethyl-N′,N′-dicarboxymethylethylenediamine, monosodium N-oleoyl-N-hydroxyethyl-N′,N′-dicarboxymethylethylenediamine, monosodium N-cocoyl-N-hydroxyethyl-N′,N′-dicarboxymethylethylenediamine, disodium N-lauroyl-N-hydroxyethyl-N′,N′-dicarboxymethylethylenediamine, disodium N-oleoyl-N-hydroxyethyl-N′,N′-dicarboxymethylethylenediamine, and disodium N-cocoyl-N-hydroxyethyl-N′,N′-dicarboxymethylethylenediamine.

Examples of (F6) semipolar surfactants include alkylamine oxide-type surfactants, alkylamine oxides, alkylamidoamine oxides, and alkyl hydroxyamine oxides. Alkyl dimethylamine oxides having from 10 to 18 carbon atoms and alkoxyethyl dihydroxyethylamine oxides having from 8 to 18 carbon atoms are preferred. Specific examples include dodecyl dimethylamine oxide, dimethyloctylamine oxide, diethyldecylamine oxide, bis-(2-hydroxyethyl) dodecylamine oxide, dipropyltetradecylamine oxide, methylethylhexadecylamine oxide, dodecylamidopropyldimethylamine oxide, cetyl dimethylamine oxide, stearyl dimethylamine oxide, tallow dimethylamine oxide, dimethyl-2-hydroxyoctadecylamine oxide, lauryl dimethylamine oxide, myristyl dimethylamine oxide, stearyl dimethylamine oxide, isostearyl dimethylamine oxide, coconut fatty acid alkyldimethylamine oxide, amidopropyl dimethylamine oxide caprylate, amidopropyl dimethylamine oxide caprate, lauric acid amidopropyl dimethylamine oxide, myristate amidopropyldimethylamine oxide, amidopropyldimethylamine palmitate, amidopropyldimethylamine oxide stearate, amidopropyldimethylamine oxide isostearate, oleic acid amidopropyldimethylamine oxide, ricinoleic acid amidopropyl dimethylamine oxide, 12-hydroxystearic acid amidopropyldimethylamine oxide, coconut fatty acid amidopropyl dimethylamine oxide, palm kernel oil fatty acid amidopropyl dimethylamine oxide, castor oil fatty acid amidopropyl dimethylamine oxide, lauric acid amidoethyl dimethylamine oxide, myristate amidoethyl dimethylamine oxide, coconut fatty acid amidoethyl dimethylamine oxide, lauric acid amidoethyl diethylamine oxide, myristate amidoethyl diethylamine oxide, coconut fatty acid amidoethyl diethylamine oxide, lauric acid amide ethyl dihydroxyethylamine oxide, myristic acid amidoethyl dihydroxyethylamine oxide, and coconut fatty acid amidoethyl dihydroxyethylamine oxide. There are no particular restrictions on the amount of the surfactant (F)

in cosmetic composition of the present invention. However, in order to stabilize the cosmetic composition, it can be blended into the cosmetic composition in a range from 0.05 to 90% by weight, preferably 0.1 to 50% by weight, and more preferably 0.5 to 25% by weight.

### (G) Powders or Colorants

A cosmetic composition of the present invention can be blended with a powder or colorant, especially a powder commonly used in cosmetic products (including powders and pigments used as colorants). A powder or colorant commonly used in cosmetics can be used without regard to shape (spherical, rod-like, acicular, tabular, sheet-like, irregular, spindle-shaped, bowl-shaped, raspberry-shaped, etc.), particle size (mist, fine particles, pigment grade, etc.), or particle structure (porous, non-porous, secondary aggregate, etc.). When these powders and/or colorants are used as a pigment, one or more selected from inorganic pigment powders, organic pigment powders, and resin powders with an average particle diameter in a range from 1 nm to 20 μm is preferred.

Examples of powders and pigments include inorganic powders, organic powders, surfactant metal salt powders (metal soaps), colored pigments, pearly pigments, metal powder pigments, and silicone elastomer powders. Compounds of these can also be used. These powders and colorants can also function as a UV protection component.

Specific examples include inorganic powders such as titanium oxide, zirconium oxide, zinc oxide, cerium oxide, magnesium oxide, barium sulfate, calcium sulfate, magnesium sulfate, calcium carbonate, magnesium carbonate, talc, mica, kaolin, sericite, muscovite, synthetic mica, phlogopite, red mica, biotite, lithium mica, silicic acid, silicic anhydride, aluminum silicate, sodium silicate, sodium magnesium silicate, magnesium silicate, aluminum magnesium silicate, calcium silicate, barium silicate, strontium silicate, metal tungstate, hydroxyapatite, vermiculite, higilite, bentonite, montmorillonite, hectorite, zeolite, ceramic powder, dibasic calcium phosphate, alumina, aluminum hydroxide, and boron nitride; organic powders such as polyamide powder, polyester powder, polyethylene powder, polypropylene powder, polystyrene powder, polyurethane powder, benzoguanamine powder, polymethylbenzoguanamine powder, polytetrafluoroethylene powder, polymethyl methacrylate powder, cellulose, silk powder, nylon powder, 12 nylon, 6 nylon, silicone powder, silicone rubber powder, silicone elastomer spherical powder coated with polymethylsilsesquioxane, polymethylsilsesquioxane spherical powder, styrene/acrylic acid copolymers, divinylbenzene/styrene copolymers, vinyl resins, urea resins, phenolic resins, fluororesins, silicon resins, acrylic resins, melamine resins, epoxy resins, polycarbonate resins, microcrystalline fiber powders, starch powder, and lauroyl lysine; surfactant metal salt powders such as zinc stearate, aluminum stearate, calcium stearate, magnesium stearate, zinc myristate, magnesium myristate, zinc palmitate, zinc laurate, zinc cetyl phosphate, calcium cetyl phosphate, and sodium zinc cetyl phosphate; colored pigments including inorganic red pigments such as red iron oxide, iron oxide, iron hydroxide and iron titanate, inorganic brown pigments such as y-iron oxide, inorganic yellow pigments such as yellow iron oxide and loess, inorganic black pigments such as black iron oxide and carbon black, inorganic violet pigments such as manganese violet and cobalt violet, inorganic green pigments such as chromium hydroxide, chromium oxide, cobalt oxide and cobalt titanate, inorganic blue pigments such as navy blue and ultramarine blue, lake tar-based pigments such as Red No. 3, Red No. 104, Red No. 106, Red No. 201, Red No. 202, Red No. 204, Red No. 205, Red No. 220, Red No. 226, Red No. 227, Red No. 228, Red No. 230, Red No. 401, Red No. 505, Yellow No. 4, Yellow No. 5, Yellow No. 202, Yellow No. 203, Yellow No. 204, Yellow No. 401, Blue No. 1, Blue No. 2, Blue No. 201, Blue No. 404, Green No. 3, Green No. 201, Green No. 204, Green No. 205, Orange No. 201, Orange No. 203, Orange No. 204, Orange No. 206 and Orange No. 207, and lake natural pigments such as carminic acid, raccaic acid, cartamine, bradylin and crocin; pearly pigments such as titanium oxide coated mica, titanium mica, iron oxide treated titanium mica, titanium oxide coated mica, bismuth oxychloride, bismuth oxychloride coated with titanium oxide, talc coated with titanium oxide, fish scale guanine, and titanium oxide-coated colored mica; and metal powder pigments such as metal powders of aluminum, gold, silver, copper, platinum, and stainless steel.

Silicone elastomer powders are the powdery component of the (L) silicone elastomers described below. These are crosslinked products of linear diorganopolysiloxanes consisting primarily of diorganosiloxy units (D units). These can be obtained by conducting a crosslinking reaction on an organohydrogenpolysiloxane having a silicon-bonded hydrogen atom in a side chain or at the terminal end and a diorganopolysiloxane having an unsaturated hydrocarbon group such as an alkenyl group in a side chain or at the terminal end in the presence of a hydrosilylation reaction catalyst. Silicone elastomer powders are softer and more elastic than silicone resin powders consisting of T units and Q units. Because they have excellent oil absorptivity, they can absorb oil on the skin and prevent makeup disintegration.

The silicone elastomer powder can take various shapes such as a spherical shape, a flat shape, or an irregular shape. The silicone elastomer powder may be in the form of an oil dispersion. The cosmetic composition of the present invention can use a silicone elastomer powder in the form of particles in which the primary particle diameter and/or average primary particle diameter measured using the laser diffraction/scattering method under observation using an electron microscope is within the range of 0.1 to 50 μm. A silicone elastomer powder having a primary particle with a spherical shape can be effectively blended. The silicone elastomer constituting the silicone elastomer powder preferably has a hardness of 80 or less, and more preferably 65 or less, as measured using a type-A durometer in accordance with JIS K6253, “Testing methods for the hardness of vulcanized rubbers and thermoplastic rubbers”.

The silicone elastomer powder can be used in a cosmetic composition of the present invention in a form of an aqueous dispersion. Commercially available products of these aqueous dispersions include BY29-129 and PF-2001 PIF Emulsion from Dow Corning Toray.

The silicone elastomer powder can be surface treated with a silicone resin or silica. Examples of surface treatments are described in JP H02-243612 A, JP H08-12545 A, JP H08-12546 A, JP H08-12524 A, JP H09-241511 A, JP H010-36219 A, JP H011-193331 A, JP 2000-281523 A, JP 2020-105330A, WO2019/124418, WO2020/137913, and WO2022/138346. Another example of silicone elastomer powders are the cross-linked silicone powders listed in the “Cosmetic Classifications and Compounding Component Standards”. Commercially available silicone elastomer powders include, for example, Tolefill E-5065, Tolefill E-508, 9701 Cosmetic Powder, and 9702 Powder from Dow Corning Toray.

Some or all of the powder or the colorant is preferably subjected to a water-repellency treatment. This enables it to be compounded stably in the oil phase. The powders or colorants may be compounded, and can be subjected to surface treatment with an all-purpose oil, a silicone compound other than an organopolysiloxane copolymer of the present invention, a fluorine compound, or a surfactant.

Examples of other water-repellent treatments include treating the powder or colorant with various water-repellent surface treatment agents. Examples include organosiloxane treatments such as methyl hydrogen polysiloxane treatment, aminosilicone treatment, silanol treatment, polyglycerin functional silicone treatment, diglycerine functional silicone treatment, silicone resin treatment, silicone gum treatment, acrylic silicone treatment and fluorinated silicone treatment, metal soap treatments such as zinc stearate treatment, silane treatments such as silane coupling agent treatment and alkyl silane treatment, fluorine compound treatments such as perfluoroalkylsilane, perfluoroalkylphosphate ester salt or perfluoropolyether treatment, amino acid treatments such as N-lauroyl-L-lysine treatment, oil treatments such as squalane treatment, and acrylic treatments such as alkyl acrylate treatment. These treatments can be used alone or in combination.

The powder or colorant is preferably treated with another powder dispersant or surface treatment agent. The dispersion or surface treatment can be performed using a novel powder treatment agent or treatment method proposed by the present inventors in WO 2009/022621 A, JP 2011-148784 A, JP 2011-149017 A, JP 2011-246704 A, JP 2011-246705 A, JP 2011-246706 A, WO 2009/022621 A, WO 2011/049246 A, WO 2011/049248 A, and Japanese Patent Application No. 2011-286973. The powder or colorant may also be slurried using one of these novel powder treatment agents or treatment methods. Because these novel treatment agents improve performance, such as improving the unique feel and dispersion stability, combined use with the novel cosmetic materials of the present invention is expected to further improve the functionality, feel, and storage stability of cosmetics.

In addition, some or all of the powder or colorant may be subjected to hydrophilic treatment. This enables the powder or colorant to be blended in relation to the aqueous phase.

In addition, some or all of the powder or colorant may be subjected to hydrophobizing and hydrophilizing treatment. This can impart emulsifying properties to the powder itself. An example of a commercially available product is MZY-500SHE from Teica.

If necessary, one or more (G) powder or colorant can be used in a cosmetic composition of the present invention. There are no particular restrictions on the amount, but they can be blended in a range from 0.1 to 99.5% by mass, and preferably from 1 to 99% by mass, relative to the entire cosmetic composition. The blending amount in the case of a solid powder cosmetic is preferably in a range from 80 to 99% by mass relative to the entire cosmetic composition.

### (H) Gelling or Thickening Agents

The gelling agent is preferably oil soluble. Specific examples include metal soaps such as aluminum stearate, magnesium stearate and zinc myristate, amino acid derivatives such as N-lauroyl-L-glutamic acid and a, y-di-n-butylamine, dextrin fatty acid esters such as dextrin palmitate, dextrin stearate and dextrin 2-ethylhexanoate palmitate, Sucrose fatty acid esters such as sucrose palmitate and sucrose stearate, and benzylidene derivatives of sorbitol such as monobenzylidene sorbitol and dibenzylidene sorbitol. These can be used alone or in combinations of two or more as needed.

### (I) Organic Modified Clay Minerals

Examples of organic modified clay minerals include dimethylbenzyl dodecylammonium montmorillonite clay, dimethyl dioctadecyl ammonium montmorillonite clay, dimethyl alkyl ammonium hectorite, benzyl dimethyl stearyl ammonium hectorite, and aluminum magnesium silicate treated with distearyl dimethyl ammonium chloride. Commercially available products include Benton 27 (benzyldimethyl stearyl ammonium chloride treated hectorite from National Red) and Benton 38 (distearyl dimethyl ammonium chloride treated hectorite from National Red).

### (J) Silicone Resins

Silicone resins are organopolysiloxanes with a highly branched, reticular or cage structure, and are liquid or solid at room temperature. Any silicone resin commonly used in cosmetics can be used as long as it does not impair the object of the present invention. Solid silicone resins include MQ resins, MDQ resins, MTQ resins, MDTQ resins, TD resins, TQ resins and TDQ combining monoorganosiloxy units (M units) (where the organo groups are only methyl groups or methyl groups and vinyl groups or phenyl groups), diorganosiloxy units (D units) (where the organo groups are only methyl groups or methyl groups and vinyl groups or phenyl groups), triorganosiloxy units (T units) (where the organo groups are methyl groups, vinyl groups or phenyl groups), and siloxy units (Q units). Examples include trimethylsiloxysilicic acid, polyalkylsiloxysilicic acid, dimethylsiloxy unit-containing trimethylsiloxysilicic acid, and alkyl (perfluoroalkyl) siloxysilicic acid. These silicone resins are oil-soluble, and those that can be dissolved in D4 and D5 are preferred. Silicone resins form a uniform film when applied to skin and hair to provide protection against drying out and against low temperatures. Silicone resins with these branch units adhere firmly to skin and hair, and impart gloss and translucence to the skin and hair.

### (K) Silicone Gums

In the present invention, an organopolysiloxane with an ultrahigh viscosity of 1,000,000 mm²/s or more is referred to as a silicone gum, but can be used as a silicone oil. Silicone gums are linear diorganopolysiloxane with a very high degree of polymerization, and are also known as raw silicone gums or organopolysiloxane gums. Silicone gums are distinguished from oily silicones in that they have a measurable degree of plasticity due to their high degree of polymerization. Examples of raw silicone gums include substituted or unsubstituted organopolysiloxanes with dialkylsiloxy units (D units) dimethylpolysiloxane, methylphenylpolysiloxane, aminopolysiloxane, and methylfluoroalkyl polysiloxane, or those with a finely crosslinked structure of these. A typical example is a compound represented by the general formula: R¹⁰(CH₃)₂SiO{(CH₃)₂SiO}ₛ{(CH₃)R¹¹SiO}₁Si(CH₃)₂R¹⁰. (In this formula, R¹¹ is a group selected from among a vinyl group, a phenyl group, an alkyl group having from 6 to 20 carbon atoms, an aminoalkyl group having from 3 to 15 carbon atoms, a perfluoroalkyl group having from 3 to 15 carbon atoms, and quaternary ammonium base-containing alkyl group having from 3 to 15 carbon atoms, a terminal R¹⁰ is a group selected from among an alkyl group having from 1 to 8 carbon atoms, a phenyl group, a vinyl group, an aminoalkyl group having from 3 to 15 carbon atoms, a hydroxyl group and alkoxy group having from 1 to 8 carbon atoms, s=2,000 to 6,000, t=0 to 1,000, and s+t=2,000 to 6,000.) Among these, a dimethylpolysiloxane raw gum with a degree of polymerization from 3,000 to 20,000 is preferred. These silicone gums can be added to a cosmetic composition of the present invention directly or in the form of a liquid gum dispersion dispersed in oily silicone (oil dispersion of silicone gum).

Examples of raw silicone gums include substituted or unsubstituted organopolysiloxanes with dialkylsiloxy units (D units) dimethylpolysiloxane, methylphenylpolysiloxane, aminopolysiloxane, and methylfluoroalkyl polysiloxane, or those with a finely crosslinked structure of these. A typical example is a compound represented by the general formula: R¹⁰(CH₃)₂SiO{(CH₃)₂SiO}ₛ{(CH₃)R¹²SiO}₁Si(CH₃)₂R¹⁰. (In this formula, R¹² is a group selected from among a vinyl group, a phenyl group, an alkyl group having from 6 to 20 carbon atoms, an aminoalkyl group having from 3 to 15 carbon atoms, a perfluoroalkyl group having from 3 to 15 carbon atoms, and quaternary ammonium base-containing alkyl group having from 3 to 15 carbon atoms, a terminal R¹⁰ is a group selected from among an alkyl group having from 1 to 8 carbon atoms, a phenyl group, a vinyl group, an aminoalkyl group having from 3 to 15 carbon atoms, a hydroxyl group and alkoxy group having from 1 to 8 carbon atoms, s=2,000 to 6,000, t=0 to 1,000, and s+t=2,000 to 6,000.) An amino-modified methylpolysiloxane raw gum having, for example, a 3-aminopropyl group or N-(2-aminoethyl) 3-aminopropyl group in a side chain or on a terminal end of the molecule is preferred. In the present invention, these silicone gums can be used alone or in combinations of two or more as needed.

Because silicone gums have a very high degree of polymerization, they form a long-lasting protective film on skin and hair with excellent breathability. This enables gloss and luster to be imparted to skin and hair, and imparts texture and firmness to skin and hair during and after use.

The amount of silicone gum added can be in a range from 0.05 to 30% by weight (mass), preferably in a range from 1 to 15% by weight (mass) % relative to the entire cosmetic composition. If a silicone gum is used in the form of an emulsified composition prepared beforehand using an emulsifying step (such as emulsion polymerization), it can be more easily and stably blended into a cosmetic composition of the present invention. If the amount of silicone gum is below the lower limit, gloss may be insufficiently imparted to skin and hair.

### (L) Silicone Elastomers

Silicone elastomers can be blended with a cosmetic composition in any form depending on the intended purpose. In addition to the silicone elastomer powders described in (G) Powders or Colorants section above, mixing a silicone elastomer in the form of a crosslinkable organopolysiloxane is preferred. A silicone elastomer powder can also be used in a cosmetic composition of the present invention in the form of an aqueous dispersion. Commercially available aqueous dispersions include, for example, BY 29-129 and PF-2001 PIF Emulsion from Dow Corning Toray. Blending in these silicone elastomer powders in the form of an aqueous dispersion (that is, a suspension) is extremely useful from the standpoint of further improving the feel of a cosmetic composition of the present invention when used.

Non-emulsifiable crosslinkable organopolysiloxanes having a structure in which an organopolysiloxane chain is three-dimensionally crosslinked in a reaction with a crosslinkable component and not having hydrophilic components such as polyoxyalkylene units are preferred. These crosslinkable organopolysiloxanes can be used without restriction, regardless of the manufacturing method such as dilution and physical form such as its properties. Especially preferred examples include the a, w-diene crosslinked silicone elastomers described in U.S. Pat. No. 5,654,362 (available commercially from Dow Corning of the US as DC 9040 Silicone Elastomer Blend, DC 9041 Silicone Elastomer Blend, DC 9045 Silicone Elastomer Blend, and DC 9046 Silicone Elastomer Blend. Crosslinkable organopolysiloxanes that have fluidity at room temperature can also be used. An example is 3901 LIQUID SATIN BLEND (Dow Chemical of the US).

### (M) Organically Modified Silicones

These organically modified silicones are preferably lipophilic. Specific examples include amino-modified silicone, amino polyether-modified silicone, epoxy-modified silicone, carboxyl-modified silicone, amino acid-modified silicone, carbinol-modified silicone, acrylic-modified silicone, phenol-modified silicone, amidoalkyl-modified silicone, aminoglycol-modified silicone, and alkoxy-modified silicone. In addition to a polysiloxane bond as a main chain, these organically modified silicones may have an alkylene chain, an aminoalkylene chain or a polyether chain to the extent that the compound does not have hydrophilicity. The organic modifying group may be in a side chain and/or on a terminal end of the polysiloxane chain. When a cosmetic composition of the present invention is a hair care product, an amino-modified silicone, carbinol-modified silicone, amino polyether-modified silicone or amino glycol-modified silicone can be used. An example is an amino-modified silicone having a 3-aminopropyl group or an N-(2-aminoethyl) 3-aminopropyl group.

The following is a description of higher alkyl-modified silicones, alkyl-modified silicone resins, and polyamide-modified silicone resins which are preferred examples of organically modified silicones. Higher alkyl-modified silicones are waxy at room temperature and are useful components as raw materials in cosmetics. As a result, they can be used advantageously in cosmetics of the present invention. Examples of higher alkyl modified silicone waxes include methyl long-chain alkylpolysiloxane capped at both ends of the molecular chain with a trimethylsiloxy group, dimethylpolysiloxane/methyl long-chain alkylsiloxane copolymers capped at both ends of the molecular chain with a trimethylsiloxy group, and long-chain alkyl-modified dimethylpolysiloxane capped at both ends of the molecular chain. Commercially available products include AMS-C30 Cosmetic Wax and 2503 Cosmetic Wax (from Dow Chemical of the US).

In a cosmetic composition of the present invention, the higher alkyl-modified silicone wax preferably has a melting point of 60° C. or more from the standpoint of longer lasting makeup and higher temperature stability.

Alkyl-modified silicone resins impart sebum resistance, moisturizing properties, and a fine texture to cosmetics. Those that are waxy at room temperature are preferred. A preferred example is the silsesquioxane resin wax described in JP 2007-532754 A. A commercially available product is SW-8005 C30 RESIN WAX (from Dow Chemical of the US).

Examples of polyamide-modified silicones include, for example, the siloxane-based polyamide compounds described in U.S. Pat. No. 5,981,680 (JP 2000-038450 A) and JP 2001-512164 A. Commercially available products include 2-8178 Gellant and 2-8179 Gellant (from Dow Chemical of the US). These polyamide-modified silicones also function as a thickening/gelling agent for oily raw materials, especially silicone oils.

### (N) UV Protection Components

UV protection components include organic UV protection components and inorganic UV protection components. When a cosmetic composition of the present invention is a sunscreen cosmetic, use of at least one type of organic or inorganic UV protection component is preferred, and use of an organic UV protection component is especially preferred. A solvent-soluble solid particle of the present invention is usually compatible with poorly soluble organic ultraviolet protective such components as hexyl diethylaminohydroxybenzoyl benzoate (Ubinal A), bisethylhexyloxyphenol methoxyphenyl triazine (Tinosorb S), 2-cyano-3,3-diphenylpropa-2-enoic acid 2-ethylhexyl ester (Octocrylene), and cinnamic acid-based UV absorbers, and these can improve the blend stability of a solvent-soluble solid particle of the present invention.

Inorganic ultraviolet protection components include inorganic pigment powders and metal powder pigments blended in the form of ultraviolet dispersants. Examples include metal oxides such as titanium oxide, zinc oxide, cerium oxide, lower titanium oxide and iron-doped titanium oxide, metal hydroxides such as iron hydroxide, metal flakes such as tabular iron oxide and aluminum flakes, and ceramics such as silicon carbide. Among these, at least one selected from among granular, tabular, acicular, or fibrous metal oxides and metal hydroxide fine particles with an average particle size in a range from 1 to 100 nm is preferred. These powders may be subjected to one or more surface treatments common in the art. Examples include fluorine compound treatments (preferably perfluoroalkyl phosphate treatment, perfluoroalkylsilane treatment, perfluoropolyether treatment, fluorosilicone treatment, and fluorinated silicone resin treatment), silicone treatments (preferably methyl hydrogen polysiloxane treatment, dimethyl polysiloxane treatment, and vapor phase tetramethyl tetrahydrogen cyclotetrasiloxane treatment), silicone resin treatment (preferably trimethylsiloxysilicic acid treatment), pendant treatments (a method in which alkyl chains etc. are added after gas phase silicone treatment), silane coupling agent treatments, titanium coupling agent treatments, silane treatments (preferably alkylsilane or alkylsilazane treatment), oil treatments, N-acylated lysine treatments, polyacrylic acid treatments, metal soap treatments (preferably with stearic acid or myristate), acrylic resin treatments, and metal oxide treatments. In another example, the surface of fine titanium oxide particles is treated with an alkylsilane after the surface of the fine particles has been coated with a metal oxide such as silicon oxide or alumina. The amount of surface treatment is preferably in a range from 0.1 to 50% by mass relative to the total mass of the powder.

Organic UV protection components are lipophilic UV protection components. Examples include benzoic acid-based UV absorbers such as p-aminobenzoic acid (PABA), PABA monoglycerin ester, N,N-dipropoxy PABA ethyl ester, N,N-diethoxy PABA ethyl ester, N,N-dimethyl PABA ethyl ester, N, N-dimethyl PABA butyl ester, and hexyl diethylaminohydroxybenzoyl benzoate; anthranilic acid-based UV absorbers such as homomenthyl-N-acetylanthranilate; salicylic acid-based UV absorbers such as amyl salicylate, menthyl salicylate, homomenthyl salicylate, octyl salicylate, phenyl salicylate, benzyl salicylate, and p-isopropanol phenyl salicylate; cinnamic acid-based UV absorbers such as octylcinnamate, ethyl-4-isopropyl cinnamate, methyl-2,5-diisopropylcinnamate, ethyl-2,4-diisopropylcinnamate, methyl-2,4-diisopropylcinnamate, propyl-p-methoxycinnamate, isopropyl-p-methoxycinnamate, isoamyl-p-methoxycinnamate, octyl-p-methoxycinnamate (2-ethylhexyl-p-methoxycinnamate), 2-ethoxyethyl-p-methoxycinnamate, cyclohexyl-p-methoxycinnamate, ethyl-a-cyano-β-phenylcinnamate, 2-ethylhexyl-a-cyano-β-phenylcinnamate, glyceryl mono-2-ethylhexanoyl-diparamethoxycinnamate, and 3-methyl-4-[methylbis (trimethylsiloxy) silyl] butyl 3,4,5-trimethoxycinnamate; benzophenone-based UV absorbers such as 2,4-dihydroxybenzophenone, 2,2′-dihydroxy-4-methoxybenzophenone, 2,2′-dihydroxy-4,4′-dimethoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4methoxybenzophenone-5-sulfonate, 4-phenylbenzophenone, 2-ethylhexyl-4′-phenyl-benzophenone-2-carboxylate, hydroxy-4-n-octoxybenzophenone, and 4-hydroxy-3-carboxybenzophenone; and others such as 3-(4′-methylbenzylidene)-d,l-camphor, 3-benzylidene-d,l-camphor, urocanic acid, urocanic acid ethyl ester, 2-phenyl-5-methylbenzoxazole, 2,2′-hydroxy-5-methylphenylbenzotriazole, 2-(2′-hydroxy-5′-t-octylphenyl)benzotriazole, 2-(2′-hydroxy-5′-methylphenyl)benzotriazole, dibenzalazine, dianisoylmethane, 4-methoxy-4′-t-butyldibenzoylmethane, and 5-(3,3-dimethyl-2-norbornylidene)-3-pentan-2-one.

These organic ultraviolet protection components can also be incorporated into a hydrophobic polymer powder. This polymer powder may be hollow, have an average primary particle size in a range from 0.1 to 50 μm, and have a broad or narrow particle size distribution. The polymer used here can be an acrylic resin, methacrylic resin, styrene resin, polyurethane resin, polyethylene, polypropylene, polyethylene terephthalate, silicone resin, nylon, acrylamide resin, or silylated polypeptide resin. A polymer powder containing an organic UV protective component in a range from 0.1 to 30% by mass is preferred, and a polymer powder containing the UV-A absorber 4-tert-butyl-4′-methoxydibenzoylmethane is especially preferred.

These organic ultraviolet protective component can also be dispersed in water. An example of such a commercially available product is Tinosorb A2B (from BASF).

In a cosmetic composition of the present invention, at least one type of ultraviolet ray protection component selected from a group consisting of fine particle titanium oxide, fine particle zinc oxide, 2-ethylhexyl paramethoxycinnamate, 4-tert-butyl-4′-methoxydibenzoylmethane, hexyl diethylaminohydroxybenzoyl benzoate, bisethylhexyloxyphenol methoxyphenyl triazine, 2-cyano-3,3-diphenylprop-2-enoic acid 2-ethylhexyl ester, and benzophenone UV absorbers can be used. These UV protection components are widely used and readily available, and have a high UV protection effect. A combination of organic and inorganic ultraviolet protection components is preferred, and a combination of an ultraviolet protection component corresponding to UV-A and an ultraviolet protection component corresponding to UV-B is especially preferred.

### (O) Water-Soluble Polymers

A cosmetic composition of the present invention may also be an aqueous emulsion containing a large amount of water-soluble components, and (O) the water-soluble polymer preferably blended in depends on the formulation. One or more water-soluble polymers can be used. Examples of water-soluble polymers include plant polymers such as gum arabic, tragacanth gum, galactan, guar gum, carob gum, karaya gum, carrageenan, pectin, agar, quince seed (quince), algae colloid (brown algae extract), starch (rice, corn, potato, wheat), and glycyrrhizic acid; microbial polymers such as xanthan gum, dextran, succinoglucan, and pullulan; and animal polymers such as collagens, caseins, albumin, and gelatins. Examples of semi-synthetic water-soluble polymers include starch-based polymers such as carboxymethyl starch and methylhydroxypropyl starch; cellulosic polymers such as methylcellulose, nitrocellulose, ethylcellulose, methylhydroxypropylcellulose, hydroxyethylcellulose, cellulose sodium sulfate, hydroxypropylcellulose, sodium carboxymethylcellulose (CMC), crystalline cellulose, and cellulose powders; and alginic acid-based polymers such as sodium alginate and propylene glycol alginate. Examples of synthetic water-soluble polymers include vinyl polymers such as polyvinyl alcohol, polyvinyl methyl ether polymer, polyvinyl pyrrolidone, and carboxyvinyl polymer (CARBOPOL 940 and 941 from BF Goodrich), polyoxyethylene polymers such as polyethylene glycol 20,000, polyethylene glycol 6,000, and polyethylene glycol 4,000; copolymers such as polyoxyethylene polyoxypropylene copolymers and PEG/PPG methyl ether; acrylic polymers such as sodium polyacrylate, polyethyl acrylate, and polyacrylamide; polyethylene imines; and cationic polymers. Other cationic water-soluble polymers that can be used especially in hair care products include quaternary nitrogen-modified polysaccharides (cation-modified cellulose, cation-modified hydroxyethyl cellulose, cation-modified guar gum, cation-modified locust bean gum, cation-modified starch, etc.), dimethyldiallylammonium chloride derivatives (such as dimethyldiallylammonium chloride/acrylamide copolymers, polydimethylmethylenepiperidinium chloride, etc.), and vinylpyrrolidone derivatives (such as vinylpyrrolidone/dimethylaminoethyl methacrylic acid copolymer salts, vinylpyrrolidone/methacrylamidopropyl trimethylammonium chloride copolymers, vinylpyrrolidone/methylvinylimidazolium chloride copolymers, etc.).

Other components commonly used in the art can be added to a cosmetic composition of the present invention in a range that does not impair the effects of the invention. Examples include organic resins, moisturizers, preservatives, antibacterial agents, fragrances, salts, antioxidants, pH adjusters, chelating agents, cooling agents, anti-inflammatory agents, skin-beautifying ingredients (whitening agents, cell activators, skin roughness improving agents, blood circulation promoters, skin astringents, antiseborrheic agents, etc.), vitamins, amino acids, nucleic acids, hormones, and inclusion compounds. Specific examples are listed in paragraphs 0100 to 0113 of JP 2011-149017 A, but the present invention is not limited to these examples.

A cosmetic composition of the present invention may be blended with natural plant extract components, seaweed extract components, and herbal components depending on the intended use. Two or more of these components may also be used. Specific examples are listed in paragraph 0115 of JP 2011-149017 A, but the present invention is not limited to these examples.

A cosmetic composition of the present invention may be blended with a solvent such as a light isoparaffin, ether, LPG, N-methylpyrrolidone, or next-generation freon in addition to purified water or mineral water depending on the intended use.

In addition to a copolymer of the present invention, a cosmetic composition of the present invention may also include at least one type selected from the group consisting of acrylic silicone dendrimer copolymers and alkyl-modified silicone resin waxes. These are film-forming components like a copolymer of the present invention but, unlike a copolymer of the present invention, they do not improve washability. Therefore, these should be used in a range that does not impair the technical effects of the present invention.

A preferred example of an acrylic silicone dendrimer copolymers is the vinyl polymer with a carbosiloxane dendrimer structure in the side chain described in Japanese Patent No. 4,009,382 (JP 2000-063225 A). Examples of commercially available products include FA 4001 CM Silicone Acrylate and FA 4002 ID Silicone Acrylate from Dow Corning Toray.

A preferred example of an alkyl-modified silicone resin wax is the silsesquioxane resin wax described in JP 2007-532754 A.

A cosmetic composition of the present invention can take the form of a liquid, emulsion, cream, solid, paste, gel, powder, multilamellar emulsion, mousse, or spray.

### [Production Method of Cosmetic Composition]

A cosmetic composition of the present invention can be produced by a method comprising a step of preparation of solution or dispersion of the solid particle described above into at least one cosmetic liquid medium.

As said cosmetic liquid medium, any cosmetic liquid medium commonly used in cosmetics can be used alone or in combination with other cosmetic liquid medium(s), as long as it does not impair the object of the present invention. Preferably, the cosmetic liquid medium is at least one selected from alcohols, esters, silicone fluid, hydrocarbon oils, fatty acid ester oils, liquid UV protection agent, and mixture thereof.

As exemplary alcohols, any alcohols disclosed in part “(E) Alcohols” can be used, as long as it does not impair the object of the present invention.

As exemplary esters, any conventional ester solvents such as methyl acetate, ethyl acetate, butyl acetate, and isobutyl acetate can be used, as long as it does not impair the object of the present invention.

As exemplary silicone fluid, any silicone oils disclosed in part “(D1) Silicone Oils” can be used, as long as it does not impair the object of the present invention.

As exemplary hydrocarbon oils, any hydrocarbon oils described above as examples of hydrocarbon oils (D2-1) can be used, as long as it does not impair the object of the present invention.

As exemplary fatty acid ester oils, any fatty acid ester oils described above as examples of fatty acid ester oils (D2-2) can be used, as long as it does not impair the object of the present invention.

As exemplary liquid UV protection agent, any agent disclosed in part “(N) UV Protection Components” can be used, as long as it does not impair the object of the present invention.

More preferably, liquid UV protection agent, or a cosmetic liquid medium mixture comprising liquid UV protection agent is anticipated to use as medium when the solid particle described above is applied/formulated into the cosmetic compositions. As exemplary liquid UV protection agent, there is OMC: ETHYLHEXYL METHOXYCINNAMATE (Uvinul® MC 80, BASF).

Further, because solvent-soluble solid particles of the present invention have superior solubility and easy handling property as cosmetic ingredient, they can be used in non-cosmetic products as well such as in external preparations, paints, coating agents, defoamers, and deodorants.

### EXAMPLES

The following is a more detailed description of the present invention with reference to examples. The present invention, however, is not restricted to these examples.

### [Calculated Glass Transition Point]

The glass transition point of the vinyl-based copolymer was calculated from a FOX equation. The FOX equation of the glass transition point (Tg) is as follows.

Tg is determined by a Fox formula (source: Radical Polymerization Handbook, P 566 (1999))

$$T g = \frac{Σ W n}{Σ Wn / Tgn}$$

(Wn: monomer weight, Tgn: Tg of a homopolymer of a monomer n, units: K)

### [Particle Size]

For emulsion, Particle size was determined as D (50) by Beckman Coulter DelsaNano C.

For solid size, size was determined by mesh size of screen (JIS Z 8801) or optical microscope.

### [Contact Angle (Water)]

An IPA solution of a vinyl based copolymer was coated onto a glass plate, after which the solvent was removed by drying at room temperature, and a coating film of a vinyl-based polymer was obtained. A 5 μl. Water droplet was placed on the coating film surface, and the contact angle with water was measured. A drop shape analysis system (KRUSS DSA10 Mk-2) was used as the measurement device, and an average value of n=5 or greater was determined.

### [Contact Angle (Artificial Sebum)]

An IPA solution of a vinyl-based copolymer was coated onto a glass plate, after which the solvent was removed by drying at room temperature, and a coating film of a vinyl-based polymer was obtained. A 5 μL droplet of artificial sebum (triolein:oleic acid:squalene=3:1:1 mixture) was placed on the coating film surface, and the contact angle with respect to the artificial sebum was measured. A drop shape analysis system (KRUSS DSA10 Mk-2) was used as the measurement device, and an average value of n=5 or greater was determined.

### [Aggregation]

After charging 1 g of sample into a 20 cc vial and sealing, the vial was placed in a 40° C. oven for 30 minutes. After that, the vial was laid on its side and the appearance of the vial was evaluated. Less than 10% of adhered amount to the inside wall was designated as “Less,” 10-25% adhered was designated as “Some,” and 50% or more was designated as “Much”.

Since many solidification processes are heat-generating or heated, agglomeration is an issue, less agglomeration is required in this test.

### [Solubility Test]

After charging 0.5 g of sample and 2 g of oil into a 20 cc vial and sealing, the vial was placed in a 50° C. oven. The portion was mixed with a dental mixer for 10 seconds every 5 minutes. Then, the time completely solved was recorded.

### [Synthesis Example 1] [Copolymer Intermediate 1 (CI-1)]

16.3 kg of isopropyl alcohol (IPA) was inserted into a 100 liter four-neck flask equipped with a stirring device, a thermometer, and a reflux tube.

The mixture was bubbled with nitrogen gas, and then sufficiently degassed and heated to 70° C. 7.6 kg (38% by weight) of methyl methacrylate (MMA), 2.4 kg (12% by weight) of n-butyl acrylate (BA), 10.0 kg (50% by weight) of a carbosiloxane dendrimer monomer as expressed by the following formula (A-1):

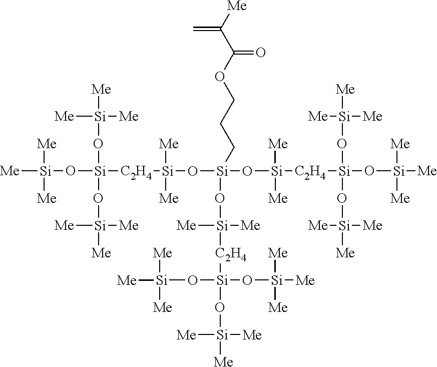

440 g (2.2% by weight) of methyl 2,2′-azobis-2-isobutyrate (V-601, manufactured by Wako Pure Chemical Industries, Ltd.), and 10.7 kg of IPA were introduced and dissolved in a dripping funnel.

In a nitrogen atmosphere, the monomer mixture was dripped through a dripping funnel over 1 hour while being maintained at 70° C. After dripping was completed, heating and stirring were performed for 8 hours under a nitrogen atmosphere to obtain a reaction product with a nonvolatile content of 40.6%.

### [Synthesis Examples 2˜7] [CF-2 to 7]

Copolymers in solvent were prepared in the same manner as Example 1 with the exception that the monomer raw materials/radical initiators/solvents and wt % of Example 1 were changed as shown in Tables 1 to 5 below. The abbreviations used in the tables are as follows: Component (A):

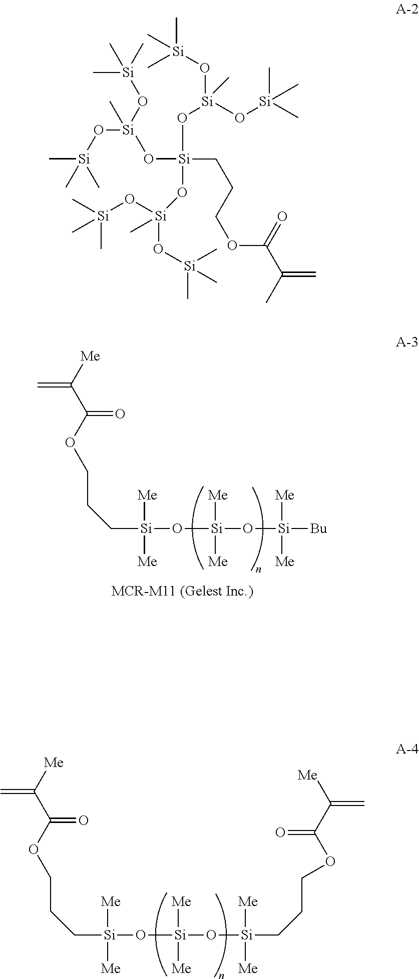

### Component (B):

BA: n-butyl acrylate
MMA: methyl methacrylate

### [Synthesis Example 8] [Copolymer Intermediate 8 (CI-8)]

### [Monomer Emulsification]

In a first beaker, 5.4 g (1.79% by weight) ECOSURF EH-40 (75% aqueous solution of 2-Ethyl Hexanol EO-PO Nonionic surfactant, Dow) and 181 g (60.35 parts by weight) of ion exchanged were weighed and agitated to form an aqueous solution. In a second beaker, 0.06 g (0.02 parts by weight) of 2-phenoxyethanol, 0.22 g (0.07 parts by weight) of 2,4-Diphenyl-4-methyl-1-pentene, 46.3 g (15.42 parts by weight) of methyl methacrylate, 4.02 g (1.34 parts by weight) of butyl acrylate, and 50.3 g (16.76 parts by weight) of A-1 were weighed and homogenized. After the mixture in the second beaker was fed to the first beaker and agitated for several minutes, the contents were passed through a pressure of 300 to 400 kg/cm² for multiple times by using a homogenizer to obtain a milky monomer emulsion. D50 was 144 nm and it was stable without any phase separation.

### [Radical Polymerization]

In a separable flask, above obtained monomer emulsion was charged and heated to 45° C. while the mixture was being agitated. After the temperature reached 45° C., 0.12 g (0.04 parts by weight) of 1% aqueous solution of Iron (II) Sulfate Heptahydrate and 0.15 g (0.05 parts by weight) of 0.1% aqueous solution Tetrasodium Ethylenediaminetetraacetate Tetrahydrate were added to the mixture in the flask. After that, 6.03 g (2.01 parts by weight) of 1.5% aqueous solution of tert-butyl hydroperoxide and 6.44 g (2.15 parts by weight) of 1.5% aqueous solution of d-isoascorbic acid were respectively and gradually added dropwise simultaneously to allow a reaction to proceed. After 2 hours, silicone acrylate aqueous dispersion (CI-8) was obtained after filtration. The non-volatile content of 1 g at 150° C. after 1 hour was 98.4%. Mw by THF-GPC was 116,000.

### [Synthesis Example 9] [Copolymer Intermediate 9 (CI-9)] [Monomer Emulsification]

In a first beaker, 2.12 g (0.705 parts by weight) of Phosten HLP-1 (90% aqueous solution of Laureth-1 phosphate, Nikko Chemicals), 1.2 g (0.4 parts by weight) of 20% sodium hydroxide aqueous solution and 167.5 g (55.825 parts by weight) of ion exchanged were weighed and agitated to form an aqueous solution. In a second beaker, 2.70 g (0.9 parts by weight) of 2-phenoxyethanol, 29.7 g (9.9 parts by weight) of methyl methacrylate, 15.3 g (5.1 parts by weight) of butyl acrylate, and 45.0 g (15 parts by weight) of A-1 were weighed and homogenized. After the mixture in the second beaker was fed to the first beaker and agitated for several minutes, the contents were passed through a pressure of 300 to 400 kg/cm² for multiple times by using a homogenizer to obtain a milky monomer emulsion.

### [Radical Polymerization]

In a separable flask, above obtained monomer emulsion was charged and heated to 80° C. while the mixture was being agitated. After the temperature reached 80° C., 22.5 g (7.5 parts by weight) of 3% of potassium persulfate aqueous solution prepared with ion exchanged water were gradually added dropwise simultaneously to allow a reaction to proceed. After 3 hours of reaction, 13.5 g (4.5 parts by weight) of 5% VA-057 (2,2′-Azobis [N-(2-carboxyethyl)-2-methylpropinamidine] tetrahydrate) aqueous solution prepared with ion exchanged water were added. After 3 hours, 0.51 g (0.17 parts by weight) of Aminopropanediol was added. Silicone acrylate aqueous dispersion (CI-9) was obtained after filtration. D50 was 98 nm and it was stable without any phase separation. The non-volatile content of 1 g at 150° C. after 1 hour was 30.8%.

### [Synthesis Example 10] [Copolymer Intermediate 10 (CI-10)]

### [Monomer Emulsification]

In a first beaker, 2.11 g (0.704 parts by weight) of Phosten HLP-1 (90% aqueous solution of Laureth-1 phosphate, Nikko Chemicals), 1.18 g (0.394 parts by weight) of 20% sodium hydroxide aqueous solution and 168.2 g (56.067 parts by weight) of ion exchanged were weighed and agitated to form an aqueous solution. In a second beaker, 1.80 g (0.6 parts by weight) of 2-phenoxyethanol, 29.7 g (9.9 parts by weight) of methyl methacrylate, 15.3 g (5.094 parts by weight) of butyl acrylate, and 44.9 g (14.983 parts by weight) of A-4 were weighed and homogenized. After the mixture in the second beaker was fed to the first beaker and agitated for several minutes, the contents were passed through a pressure of 300 to 400 kg/cm² for multiple times by using a homogenizer to obtain a milky monomer emulsion.

### [Radical Polymerization]

In a separable flask, above obtained monomer emulsion was charged and heated to 80° C. while the mixture was being agitated. After the temperature reached 80° C., 22.5 g (7.5 parts by weight) of 3% of Potassium Peroxodisulfate aqueous solution prepared with ion exchanged water were gradually added dropwise simultaneously to allow a reaction to proceed. After 3 hours of reaction, 13.5 g (4.5 parts by weight) of 5% VA-057 (2,2′-Azobis [N-(2-carboxyethyl)-2-methylpropinamidine] tetrahydrate) aqueous solution prepared with ion exchanged water were added. After 3 hours, 0.54 g (0.18 parts by weight) of Aminopropanediol was added. Silicone acrylate aqueous dispersion (CI-10) was obtained after filtration. D50 was 129 nm and it was stable without any phase separation.

TABLE 1
Copolymer intermediates (CI samples)

| Polymer in | | | | | | | | | | | |
|---|---|---|---|---|---|---|---|---|---|---|---|
| solvent/water | | CI-1 | CI-2 | CI-3 | CI-4 | CI-5 | CI-6 | CI-7 | CI-8 | CI-9 | CI-10 |
| Silicone | A-1, wt % | 50 | 0 | 30 | | | 40 | 50 | 50 | 50 | |
| monomers, | A-2, wt % | | | | | 50 | | | | | |
| wt % | A-3, wt % | | | | 50 | | | | | | |
| | A-4, wt % | | | | | | | | | | 50 |
| Non-Silicone | MMA, wt % | 38 | 65 | 45 | 38 | 38 | 55 | 45.8 | 46 | 33 | 33 |
| monomers, | BA, wt % | 12 | 35 | 25 | 12 | 12 | 5 | 4.2 | 4 | 17 | 17 |
| wt % | | | | | | | | | | | |
| Chain | 2,4-Diphenyl- | | | | | | | | 0.22 | | |
| transfer | 4-methyl-1- | | | | | | | | | | |
| agent | pentene | | | | | | | | | | |
| Radical | V-601 | 1 | 1 | | 1 | 1 | 8 | 8 | | | |
| initiator | AIBN | | | 0.3 | | | | | | | |
| | t-BHP | | | | | | | | 0.09 | | |
| | Potassium | | | | | | | | | 0.225 | 0.225 |
| | Peroxodisulfate | | | | | | | | | | |
| | VA-057 | | | | | | | | | 0.225 | 0.225 |
| Carrier | IPA | 150 | | | 150 | 150 | 150 | 150 | | | |
| | EA | | 150 | | | | | | | | |
| | toluene | | | 150 | | | | | | | |
| | Water | | | | | | | | Up-to | Up-to | Up-to |
| | | | | | | | | | 180 | 333 | 300 |
| Emulsifier | ECOSURF | | | | | | | | 5.33 | | |
| | EH-40 | | | | | | | | | | |
| | Phosten HLP-1 | | | | | | | | | 0.705 | 0.705 |
| Others | Phenoxyethanol | | | | | | | | 0.06 | 0.6 | 0.6 |
| | EDTA•4H2O | | | | | | | | 150 ppm | | |
| | Iron(II) Sulfate | | | | | | | | 0.0012 | | |
| | IAA | | | | | | | | 0.096 | | |
| | KOH | | | | | | | | | 0.008 | 0.008 |
| | AMPD | | | | | | | | | 0.18 | 0.18 |

| | | | | | | | | | | |
|---|---|---|---|---|---|---|---|---|---|---|
| Mw (peak top) | 42,800 | 54,400 | 58,700 | 39,200 | 30,300 | 13,400 | 13,100 | >500,000 | 100,900 | Not |
| | | | | | | | | | | measured |

| | | | |
|---|---|---|---|
| Process | Polymerization | Solution Polymerization | Mini-emulsion polymerization |

V-601: methyl 2,2′-azobis-2-isobutyrate (Fujifilm Wako Chemical Corporation)
AIBN: 2,2′-Azobis (isobutyronitrile) (Fujifilm Wako Chemical Corporation)
t-BHP: Luperox TBH70X (70 wt. % in H₂O, Sigma-Aldrich)
VA-057:2,2′-Azobis [N-(2-carboxyethyl)-2-methylpropinamidine] tetrahydrate (Fujifilm Wako Chemical Corporation)
Potassium Peroxodisulfate: Potassium Peroxodisulfate (Fujifilm Wako Chemical Corporation)
ECOSURF EH-40: 75% aqueous solution of 2-Ethyl Hexanol EO-PO Nonionic surfactant (Dow Chemical)
Phosten HLP-1: 90% aqueous solution of Laureth-1 phosphate (Nikko Chemicals)
EDTA. 4H₂O: Tetrasodium Ethylenediaminetetraacetate Tetrahydrate (Tokyo Chemical Industry Co., Ltd.)
IAA: d-isoascorbic acid (Sigma-Aldrich)
KOH: sodium hydroxide (Fujifilm Wako Chemical Corporation)
AMPD: 2-Amino-2-methyl-1,3-propanediol (Fujifilm Wako Chemical Corporation)

### Practical Example 1

### [Solid Copolymer 1] (Milled)

100 g of Copolymer intermediate 1 (CI-1) was charged to one-neck flask. The flask was set to rotary evaporator equipped with vacuum pump. IPA, residual monomers and volatiles in CI-1 were removed at 110-130° C. by vacuum stripping. After that, 39 g of solid polymer block was obtained. The polymer block was milled to small beads by crush mill (Osaka Chemical Wonder, Crusher WC-3). 28 g of 250-500 μm beads was obtained after screen.

### Practical Example 2

### [Solid Copolymer 2] (Small Pellet)

Prior to the trial, polymer concentration of CI-1 was adjusted to 40 wt. % conc.

A pelletizing process was constructed by coupling an extruder and a strand cutter. A strand bath was set between the extruder and the strand cutter to cool the molten strand down. 20 kg of the CI-1 (8 kg as a solid) was constantly fed with 5 kg/h into the extruder which was heated up to 100 deg.C. After volatiles were removed in the extruder, molten copolymer was extruded from the extruder with a strand die having holes of 1.5 mm in diameter. The molten was solidified as going through the strand bath where 25 deg.C water was applied. After the strand cutter was applied, pellet samples were obtained with the form of φ1 mm*3-5 mm length.

### [Practical Example 3] [

### Solid Copolymer 3] (Spray Dry)

Prior to the trial, polymer concentration of CI-1 was adjusted to 32 wt. % conc.

Spray dryer was built with air flow feed rate=3.5 Nm3/min and inlet temperature=50° C. 500 g of 32% of CI-1 (160 g as a solid) was loaded into liquid component tank, which was fed into two-fluid nozzle with feed rate of 2.0 kg/hr. IPA feed rate was determined to keep IPA vapor concentration below 20% of % LEL. Resultant outlet temperature was 28-31° C. After that, 133 g of a solid was obtained. Optical microscopy showed the primary powder size was 20-30 μm.

### Comparative Example 1

### [Solid Copolymer CE1] (Large Pellet)

Prior to the trial, polymer concentration of CI-1 was adjusted to 40 wt. % conc.

A pelletizing process was constructed by coupling an extruder and a strand cutter. A strand bath was set between the extruder and the strand cutter to cool the molten strand down. 20 kg of the CI-1 (8 kg as a solid) was constantly fed with 5 kg/h into the extruder which was heated up to 100 deg.C. After volatiles were removed in the extruder, molten copolymer was extruded from the extruder with a strand die having holes of 5 mm in diameter. The molten was solidified as going through the strand bath where 25 deg.C water was applied. After the strand cutter was applied, pellet samples were obtained with the form of φ4 mm*10 mm length.

Prior to the trial, concentration of CI-1 was adjusted to 40 wt. % conc. with IPA.

### [Comparative Example 2] [

### Solid Copolymer CE2] (Mill)

100 g of CI-2 was charged to one-neck flask. The flask was set to rotary evaporator equipped with vacuum pump. Ethyl acetate, residual monomers and volatiles in CI-2 were removed at 110-130° C. by vacuum stripping. After that, 39 g of solid polymer block was obtained. The polymer block was milled to small beads by crush mill (Osaka Chemical Wonder, Crusher WC-3). 20 g of 250-500 μm beads was obtained after screen.

### Comparative Example 3

### [Solid Copolymer CE3]

100 g of CI-3 was charged to one-neck flask. The flask was set to rotary evaporator equipped with vacuum pump. Toluene, residual monomers and volatiles in CI-3 were removed at 110-130° C. by vacuum stripping. After that, 38.5 g of solid polymer block was obtained. The polymer block was milled to small beads by crush mill (Osaka Chemical Wonder, Crusher WC-3). 14.2 g of 250-500 μm beads was obtained after screen. Some aggregated block was left on the screen.

### [Consideration 1]

### [Size]

From comparison between PE1, PE2, PE3 and CE1, larger size could not be solved in isododecane within 30 min. or shorter time. As the ease of solubility influences productivity and energy consumption, smaller size than 10 mm as longest side was good.

### [Si % and Tg]

From comparison between PE1 and CE2, No Si % could not be solved in isododecane. In addition, repellent performance was very low, which indicates long lasting was not enough.

From comparison between PE1 and CE3, low Tg caused aggregation in the aggregation test. Since solidification process requires low aggregation, a higher Tg is required.

TABLE 2
Solid polymer samples 1

| | | CE1 | CE2 | CE3 | PE1 | PE2 | PE3 |
|---|---|---|---|---|---|---|---|
| Polymer in | | CI-1 | CI-2 | CI-3 | CI-1 | CI-1 | CI-1 |
| solvent/ | | | | | | | |
| water | | | | | | | |
| Si % | | 50% | 0% | 30% | 50% | 50% | 50% |
| Calc'd Tg (Fox), ° C. | | 48 | 28 | 27 | 48 | 48 | 48 |
| Particle Size | | φ4 mm × | 250-500 μm | 250-500 μm | 250-500 μm | φ1 mm × | 20-30 μm |
| | | 10 mm | | | | 4 mm | |
| Crosslink | | No | No | No | No | No | No |
| Process | Polymerization | Sol'n | Sol'n | Sol'n | Sol'n | Sol'n | Sol'n |
| | | Polymerization | Polymerization | Polymerization | Polymerization | Polymerization | Polymerization |
| | Solidification | Evaporation | Evaporation | Evaporation | Evaporation | Evaporation | Spray dry |
| | Process | by TSE | Mill Screen | Mill Screen | Mill Screen | by TSE | |
| | | Pelletizer | | | | Pelletizer | |
| Particle Size | | φ4 mm × | 250-500 μm | 250-500 μm | 250-500 μm | φ1 mm × | 20-30 μm |
| | | 10 mm | | | | 3-5 mm | |
| Calc'd Tg (Fox), ° C. | | 48 | 28 | 27 | 48 | 48 | 48 |
| Aggregation | | Less | Less | Some | Less | Less | Less |
| Contact angle (water), ° | | 106 | 84 | 101 | 106 | 106 | 106 |
| Contact angle | | 59 | 20 | 21 | 59 | 59 | 59 |
| (artificial sebum), ° | | | | | | | |
| Solubility | IDD | Not completely | Not solved | Solved | Solved | Solved | Solved |
| test | | solved | 50° C. × | 50° C. × | 50° C. × | 50° C. × | Mixing at R.T. |
| | | 50° C. × | 30 min | 30 min | 10 min | 15 min | |
| | | 30 min | | | | | |

(PE: Practical Example, CE: Comparable Example)

### Practical Example 4-7

Samples were prepared in the same manner as practical example 1 or 3 with the exception that the copolymer intermediates, wt %, and solidification process of Example 1 were changed as shown in Table3 below. Comparable example 2 was same as described above.

### [Consideration 2]

### [Silicone Moiety]

From comparison between PE4-PE7, various silicone structures enabled easily soluble in various oils applicable to cosmetics. At this moment, polyacrylate without silicone moiety was not soluble in those oils. Silicone contributed to compatibility with various oils.

TABLE 3
Solid polymer samples 2

| | | PE4 | PE5 | PE6 | PE7 | CE2 |
|---|---|---|---|---|---|---|
| Copolymer Intermediates | | CI-4 | CI-5 | CI-6 | CI-7 | CI-2 |
| Si % | | 50% | 50% | 40% | 50% | 0% |
| | | (A-3) | (A-2) | (A-1) | (A-1) | |
| Calc'd Tg (Fox), ° C. | | 48 | 48 | 83 | 84 | 28 |
| Particle Size | | 250-500 μm | 250-500 μm | 20-30 μm | 250-500 μm | 250-500 μm |
| Crosslink | | No | No | No | No | No |
| Process | Polymerization | Sol'n | Sol'n | Sol'n | Sol'n | Sol'n |
| | | Polymerization | Polymerization | Polymerization | Polymerization | Polymerization |
| | Solidification | Evaporation | Evaporation | Spray dry | Evaporation | Evaporation |
| | Process | Mill Screen | Mill Screen | | Mill Screen | Mill Screen |
| Particle Size | | 250-500 μm | 250-500 μm | 20-30 μm | 250-500 μm | 250-500 μm |
| Calc'd Tg (Fox), ° C. | | 48 | 48 | 83 | 84 | 28 |
| Aggregation | | Less | Less | Less | Less | Less |
| Contact angle (water), ° | | 111 | 108 | 111 | 109 | 84 |
| Contact angle | | 54 | 57 | 51 | 60 | 20 |
| (artificial sebum), ° | | | | | | |
| Solubility | EtOH | Mixing | Mixing | 50° C. × | Mixing | Not solved |
| test | | at R.T. | at R.T. | 25 min | at R.T | after 30 min |
| | SH 245 | 50° C. × | 50° C. × | 50° C. × | 50° C. × | Not solved |
| | | 20 min | 10 min | 25 min | 10 min | after 30 min |
| | PDMS, 2 cst | 50° C. × | 50° C. × | Not solved | 50° C. × | Not solved |
| | | 10 min | 15 min | after 30 min | 30 min | after 30 min |
| | FZ-3196 | 50° C. × | 50° C. × | 50° C. × | 50° C. × | Not solved |
| | | 10 min | 20 min | 25 min | 30 min | after 30 min |
| | SH 556 | 50° C. × | 50° C. × | Not solved | 50° C. × | Not solved |
| | | 30 min | 30 min | after 30 min | 30 min | after 30 min |
| | IDD | Mixing | Mixing | 50° C. × | 50° C. × | Not solved |
| | | at R.T. | at R.T. | 10 min | 10 min | after 30 min |
| | Cetiol ultimate | 50° C. × | Not solved | 50° C. × | 50° C. × | Not solved |
| | | 10 min | after 30 min | 10 min | 10 min | after 30 min |
| | TKG | 50° C. × | 50° C. × | Mixing | 50° C. × | Not solved |
| | | 30 min | 30 min | at R.T | 30 min | after 30 min |
| | OMC | 50° C. × | 50° C. × | Not solved | 50° C. × | Not solved |
| | | 30 min | 30 min | after 30 min | 30 min | after 30 min |
| | Butyl Acetate | Mixing | Mixing | Not solved | Mixing | Not solved |
| | | at R.T | at R.T. | after 30 min | at R.T. | after 30 min |

(PE: Practical Example, CE: Comparable Example)

IPA: 2-propanol (Fujifilm Wako Chemical Corporation)
EA: ethyl acetate (Fujifilm Wako Chemical Corporation)
EtOH: ethanol (Fujifilm Wako Chemical Corporation)
SH 245: Decamethylcyclopentasiloxane (DOWSIL™ SH 245 Fluid, Dow Toray Co., Ltd.)
PDMS, 2cst: Dimethicone (DOWSIL™ SH 200 C Fluid 2cs, Dow Toray Co., Ltd.)
FZ-3196: Caprylyl Methicone (DOWSIL™ FZ-3196 Fluid, Dow Toray Co., Ltd.)
SH 556: Phenyl Trimethicone (DOWSIL™ SH 556 Fluid, Dow Toray Co., Ltd.)
IDD: Isododecane (PUROLAN IDD, LANXESS Distribution GmbH)
Cetiol ultimate: Undecane and Tridecane (Cetiol® Ultimate, BASF Japan)
TKG: CAPRYLIC/CAPRIC TRIGLYCERIDE (FineNeo-MCT, Nippon Fine Chemical)
OMC: ETHYLHEXYL METHOXYCINNAMATE (Uvinul® MC 80, BASF)
Butyl acetate: Butyl acetate (Fujifilm Wako Chemical Corporation)

### Practical Example 8-9

Samples were prepared after the same manner as practical example 1 or 3 with the exception that the copolymer intermediates, wt %, and solidification process of Example 1 were changed as shown after Tables 3 below. Comparable example 2 was same as described above.

### [Consideration 3]

### [Crosslinking]

Crosslinking copolymer resulted from two or more (meth)acryl functional silicones could not be prepared after solution polymerization because of gelled. Mini-emulsion polymerization was taken (CI-10). From comparison between PE8-PE9 and CE4, crosslinking structure could not make film after drying and could not be solved after isododecane, SH 556 and TKG.

TABLE 4
Solid polymer samples 3

| | | PES | PE9 | CE4 |
|---|---|---|---|---|
| Polymer after | | CI-8 | CI-9 | CI-10 |
| solvent/water | | | | |
| Si % | | 50% (A-1) | 50% (A-1) | 50% (A-4) |
| Calc'd Tg (Fox), ° C. | | 48 | 84 | 48 |
| Particle Size | | 20-30 μm | 10 μm | 20-30 μm |
| Crosslink | | No | No | Yes |
| Process | Polymerization | Mini-emulsion | Mini-emulsion | Mini-emulsion |
| | | Polymerization | Polymerization | Polymerization |
| | Solidification | Spray Dry | Spray Dry | Spray Dry |
| | Process | | | |
| Particle Size | | 20-30 μm | 10 μm | 20-30 μm |
| Calc'd Tg (Fox), ° C. | | 48 | 84 | 48 |
| Aggregation | | Less | Less | Less |
| Contact angle (water), ° | | 112 | 110 | Not measured |
| Contact angle (artificial sebum), ° | | 79 | 54 | Not measured |
| Solubility test | IDD | Solved | Solved | Not solved |
| | | 50° C. × 30 min | 50° C. × 30 min | |
| | SH556 | Solved | Solved | Not solved |
| | | 50° C. × 20 min | 50° C. × 10 min | |
| | TKG | Solved | Solved | Partially |
| | | 50° C. × 20 min | 50° C. × 10 min | solved |

### Cosmetic Formulation Examples

[Practical Example 10] Sun Care Spray

| Trade name | INCI name | Wt. % |
|---|---|---|
| Solid polymer PE6 | | 5 |
| Alcohol | Alcohol | 56.5 |
| Z Cote HP 1 | Zinc Oxide (and) | 10 |
| | Dimethicone | |
| T-Lite SF | Titanium Dioxide (and) | 10 |
| | Aluminum Hydroxide | |
| | (and) Dimethicone/ | |
| | Methicone copolymer | |
| Sunsphere ™ Bio | Microcrystalline | 1.5 |
| SPF Booster | cellulose | |
| CARBOWAX ™ SENTRY | PEG-6 | 5 |
| PEG 300 | | |
| Tinosorb S | Bis-Ethylhexyloxyphenol | 2 |
| | Methoxyphenyl Triazine | |
| Neo Heliopan AV | Ethylhexyl | 5 |
| | Methoxycinnamate | |
| Neo Heliopan OS | Ethylhexyl Salicylate | 5 |

Process: Mix all ingredients together

[Practical Example 11] Loose powder with active

| Trade name | INCI name | Wt. % |
|---|---|---|
| Solid polymer PE6 | | 5 |
| Rose Oil | Rosa Damascena Flower Oil | 5 |
| Rice Husk Silica | Silica | 5 |
| DOWSIL ™ 9608 | Dimethicone Crosspolymer | 5 |
| Cosmetic Powder | (and) Dimethicone | |
| Pearl Powder | Pearl Powder | 5 |
| Rose Powder | Rose Pollen | 0.5 |
| Licorice powder | Glycyrrhiza Uralensis | 0.5 |
| | (Licorice) Root Powder | |
| Starch Powder | Amylopectin | 30 |
| Talc | Talc | 44 |

Process: Mix all ingredients together

[Practical Example 12] Sun Care Powder

| Trade name | INCI name | Wt. % |
|---|---|---|
| Solid polymer PE6 | | 5 |
| Biobased | Isododecane | 5 |
| Z Cote HP 1 | Zinc Oxide (and) | 15 |
| | Dimethicone | |
| T-Lite SF | Titanium Dioxide (and) | 15 |
| | Aluminum Hydroxide | |
| | (and) Dimethicone/ | |
| | Methicone copolymer | |
| Sunsphere ™ Bio | Microcrystalline | 1.5 |
| SPF Booster | cellulose | |
| Starch Powder | Amylopectin | 20 |
| Talc | Talc | 33.5 |

Process: Mix all ingredients together

[Practical Example 13] Bling Powder,
Hot powder, Volume up hair powder

| Trade name | INCI name | Wt. % |
|---|---|---|
| Solid polymer PE6 | | 10 |
| Mica | Mica | 10 |
| Alcohol | Alcohol | 80 |

Process: Mix all ingredients together

[Practical Example 14] Antiperspirantand Deodorant powder

| Trade name | INCI name | Wt. % |
|---|---|---|
| Solid polymer PE6 | | 10 |
| Deodorant salt | Aluminum hydroChloride | 20 |
| | Corn Starch | 30 |
| | Kaolin | 30 |
| | Zinc Oxide | 10 |

Process: Mix all ingredients together

[Practical Example 15] Antiperspirant and Deodorant powder

| Trade name | INCI name | Wt. % |
|---|---|---|
| Solid polymer PE6 | | 10 |
| Deodorant salt | Aluminum hydroChloride | 20 |
| | Corn Starch | 70 |

Process: Mix all ingredients together

[Practical Example 16] Antiperspirant and Deodorant spray

| Trade name | INCI name | Wt. % |
|---|---|---|
| Solid polymer PE6 | | 10 |
| Deodorant salt | Aluminum hydroChloride | 20 |
| | Alcohol | 70 |

Process: Mix all ingredients together

[Practical Example 17] Pressed powder

| Trade name | INCI name | Wt. % |
|---|---|---|
| Solid polymer PE6 | | 5 |
| DOWSIL ™ 9608 | Dimethicone Crosspolymer | 10 |
| Cosmetic Powder | (and) Dimethicone | |
| SA-TR-10 | Titanium Dioxide (and) | 8.4 |
| | Dimethylpolysiloxane | |
| SA-YP-10 | Yellow Iron Oxide (and) | 2 |
| | Methylpolysiloxane | |
| SA-RPS-10 | Red Iron Oxide (and) | 0.5 |
| | Dimethylpolysiloxane | |
| SA-BP-10 | Black Iron Oxide (and) | 0.1 |
| | Methylpolysiloxane | |
| Talc | Talc | 74 |

Process: Mix all ingredients together
Add certaafter amount of powder into molder and press

[Practical Example 18] Color Ink

| Trade name | INCI name | Wt. % |
|---|---|---|
| Solid polymer PE6 | | 10 |
| UNIPURE RED LC381 | CI 77491 | 10 |
| UNIPURE RED LC3075 | CI 15850 | 10 |
| IDD | Isododecane | 35 |
| XIAMETER ™ PMX-200 Fluid, 2 cst | dimethicone | 35 |

Process: Mix all ingredients together
Similar formulations can be used after temporary hair colorant Spray

[Practical Example 19] Acne treatment gel

| Trade name | INCI name | Wt. % |
|---|---|---|
| Solid polymer PE6 | | 5 |
| SALICYLIC ACID | Salicylic Acid | 2 |
| ETHYLHEXYL PALMITATE | Ethylhexyl Palmitate | 5 |
| Caffeine | Caffeine | 0.01 |
| Hydrogenated Palm | Hydrogenated Palm | 10 |
| Kernel Oil | Kernel Oil | |
| IDD | Isododecane | 40 |

Process: Mix all ingredients together

[Practical Example 20] Anti-aging skafter lifting essence

| Trade name | INCI name | Wt. % |
|---|---|---|
| Solid polymer PE6 | | 15 |
| XIAMETER ™ PMX-200 | Hexamethyldisiloxane | 34.98 |
| Fluid, 0.65 cst | | |
| Palmitoyl oligopeptide | Palmitoyl Tripeptide-1 | 0.01 |
| Caffeine | Caffeine | 0.01 |
| Hydrogenated Palm | Hydrogenated Palm | 10 |
| Kernel Oil | Kernel Oil | |
| IDD | Isododecane | 40 |

Process: Mix all ingredients together

[Practical Example 21] Fragrance Spray/Gel/ointment/stick

| Trade name | INCI name | Wt. % |
|---|---|---|
| Solid polymer PE6 | | 15 |
| fragrance | | 5 |
| Carbitol ™ JSQI | Ethoxydiglycol | 10 |
| Alcohol | Alcohol | 70 |

Process: Mix all ingredients together

[Practical Example 22] W/O cream

| Phase | Trade name | INCI name | Wt. % |
|---|---|---|---|
| A | Solid polymer PE6 | | 5 |
| | DOWSIL ™ ES-5600 | Cetyl Diglyceryl | 2 |
| | Silicone Glycerol | Tris(Trimethyl- | |
| | Emulsifier | siloxy)silylethyl | |
| | | Dimethicone | |
| | GTCC | Caprylic Capric | 5 |
| | | Triglycerides | |
| | DOWSIL ™ EL-7040 | Caprylyl Methicone (and) | 5 |
| | Hydro Elastomer | PEG-12 Dimethicone/ | |
| | Powder | PPG-20 Crosspolymer | |
| | Jojoba Oil | Jojoba oil | 5 |
| B | Glycerine | Glycerine | 10 |
| | Water | Water | 67.5 |
| | PHENOXYETHANOL | Phenoxyethanol | 0.5 |

| | |
|---|---|
| Process: | Mix all ingredients after phase A together. |
| | Mix all ingredients after phase B together. |
| | Add phase B to phase A under mixing until |
| | homogenous cream got. |

[Practical Example 23] O/W cream

| Phase | Trade name | INCI name | Wt. % |
|---|---|---|---|
| A | Solid polymer PE6 | | 3 |
| | DOWSIL ™ ES-5373 | PEG-12 | 2 |
| | Formulation Aid | Dimethicone | |
| | ISOPAR ™ L FLUID | Isododecane | 10 |
| | DOWSIL ™ FZ-3196 Fluid | Caprylyl Methicone | 5 |
| B | ACULYN ™ Siltouch | Sodium Acrylate/Sodium | 1 |
| | Rheology Modifier | Acryloyldimethyl | |
| | | Taurate Copolymer | |
| | | (and) Dimethicone | |
| | | (and) Trideceth-6 (and) | |
| | | PEG/PPG-18/18 | |
| | | Dimethicone | |
| | water | Water | To 100 |
| | Glycerine | Glycerine | 5 |
| | Fragrance | Fragrance | 0.5 |
| | PHENOXYETHANOL | Phenoxyethanol | 0.5 |

Mix all ingredients after phase A together.
Mix all ingredients after phase B together.
Mix phase A and B under high-speed mixing.

1. A solid particle consisting essentially of organosilicon-functional co-polymer polymerized from a monomer composition consisting essentially of:
(A) one or more unsaturated polymerizable monomer(s) having at least one organosilicon functionality and one polymerizable group in the molecule; and
(B) one or more unsaturated polymerizable monomer(s) containing no or one silicon atom and having one polymerizable group in the molecule;
wherein the mass ratio of monomer (A) to monomer (B) ranges from 35:65 to 70:30 in the monomer composition, and the long size in three directions for the solid primary particle ranges from 0.1 to 5,000 μm.

2. The solid particle according to claim 1, wherein the glass transition point (Tg), which is calculated from a FOX equation, of the solid particle ranges from 35 to 120° C.

3. The solid particle according to claim 1, wherein the unsaturated polymerizable monomer (A) is at least one selected from one represented by any of following formulae (A-1) to (A-7): 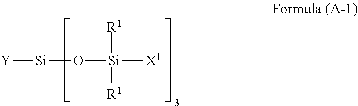
where Y is a radically polymerizable organic group, R¹ is an alkyl or aryl group having from 1 to 10 carbon atoms, and X¹ is a silylalkyl group represented by the following formula where i=1; 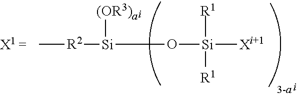
where R¹ is the same as above, R² is an alkylene group having from 2 to 10 carbon atoms, R³ is an alkyl group having from 1 to 10 carbon atoms, Xⁱ⁺¹ is a hydrogen atom or a group selected from the group consisting of an alkyl group having from 1 to 10 carbon atoms, an aryl group, and a silylalkyl group mentioned above, i is an integer from 1 to 10 representing the number of levels of silylalkyl groups mentioned above, and aⁱ is an integer from 0 to 3; 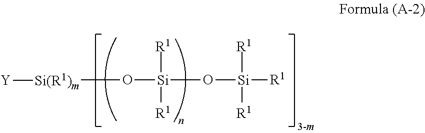
where Y and R¹ are the same as above, m is 0, 1 or 2, and n is a number from 0 to 200 representing the average degree of polymerization; 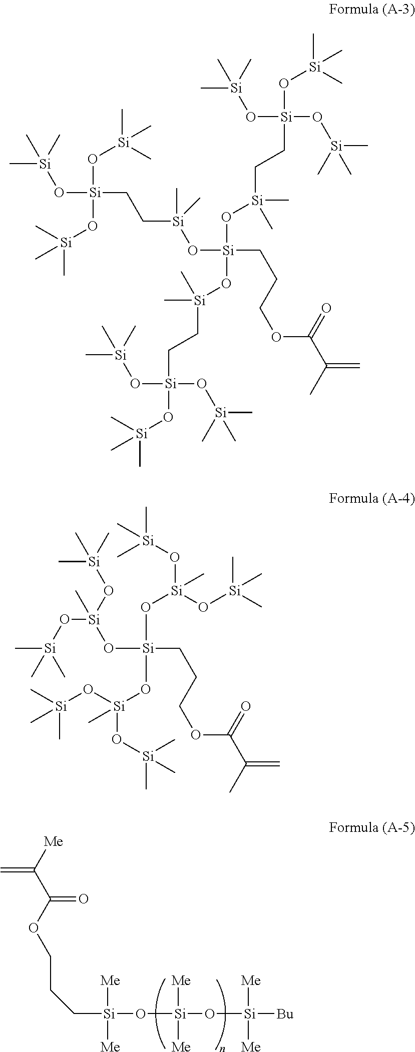
where n is a number from 0 to 120;

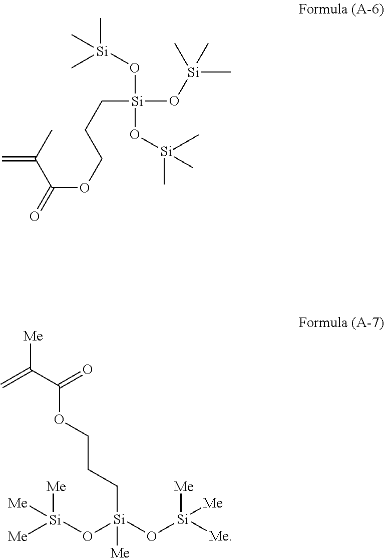

4. The solid particle according to claim 1, wherein the shape of the solid particle is selected from a spherical particle, a non-spherical particle, a powder, a pellet, a bead, a short fiber, a short tube, and a crushed powder.

5. The solid particle according to claim 1, wherein the shape of the solid primary particle is a spherical particle having a diameter ranging from 0.1 to 5,000 μm.

6. A manufacturing process of the solid particle according to claim 1, the process comprising following steps:
preparing a solution or dispersion of organosilicon-functional co-polymer through polymerization reaction from the monomer composition; and
removing carrier fluid of water or solvent from the solution or dispersion of organosilicon-functional co-polymer prepared in step (I).

7. The manufacturing process according to claim 6, further comprising:
forming the solid particle consisting essentially of organosilicon-functional co-polymer using at least one device selected from an extruder, a pelletizer, a mill, a crusher, a pulverizer, a grinder, a pastillator, and a drum flaker, following step.

8. The manufacturing process according to claim 7, further comprising:
classifying the solid particle using at least one device selected from a screen filter, a mesh, a punch plate, and a cyclone and dynamic air classifier.

9. The manufacturing process according to claim 6, wherein step (II) is a spray-drying process to obtain spherical particle of the organosilicon-functional co-polymer by removing carrier fluid of water or solvent from the solution or dispersion of organosilicon-functional co-polymer through spraying the solution or dispersion.

10. The manufacturing process according to claim 6, wherein step (I) is a step of preparing a solution or dispersion of organosilicon-functional co-polymer through at least one liquid phase polymerization reaction selected from solution polymerization, mini-emulsion polymerization, and emulsion polymerization.

11. The solid particle according to claim 1, further defined as a cosmetic ingredient.

12. The solid particle according to claim 11, wherein the cosmetic ingredient has film-forming function in human skin and/or hair.

13. A cosmetic composition comprising the solid particle according to claim 1.

14. A production method of the cosmetic composition of claim 13, the method comprising preparing a solution or dispersion of the solid particle into at least one cosmetic liquid medium.

15. The production method according to claim 14, wherein the cosmetic liquid medium is at least one selected from alcohols, esters, silicone fluids, hydrocarbon oils, fatty acid ester oils, liquid UV protection agents, biobased liquids, biodegradable liquids, cosmetically-acceptable solvents, and mixtures thereof.
